(19) United States
(12) Patent Application Publication
(10) Pub. No.: US 2024/0166594 A1
(43) Pub. Date: May 23, 2024

(54) PHOTOCAGED CITRULLINE ANALOGS AND METHODS FOR SITE-SPECIFIC INCORPORATION OF CITRULLINE INTO PROTEINS

(71) Applicant: University of Massachusetts, Boston, MA (US)

(72) Inventors: Paul R. Thompson, Wellesley, MA (US); Santanu Mondal, Shrewsbury, MA (US)

(21) Appl. No.: 18/279,931

(22) PCT Filed: Mar. 11, 2022
(86) PCT No.: PCT/US22/19973

§ 371 Date: Sep. 1, 2023

Related U.S. Application Data
(60) Provisional application No. 63/161,918, filed on Mar. 16, 2021.

(51) Int. Cl. C07C 275/24; A61K 38/00; C07D 311/18; C07F 5/02; C07K 1/107; C07K 2/00; C12P 21/00

(52) U.S. Cl. CPC C07C 275/24; C07D 311/18; C07F 5/022; C07K 1/1075; C07K 2/00; C12P 21/00; A61K 38/00

(57) ABSTRACT

The invention provides novel compounds and methods for site-specific incorporation of citrulline into peptides and proteins in mammalian cells. The invention also relates to modified peptides and proteins with site specific citrullination and pharmaceutical compositions and methods of preparation and use thereof.

### PRIORITY CLAIMS AND RELATED APPLICATIONS

This application claims the benefit of priority to U.S. Provisional Application No. 63/161,918, filed Mar. 16, 2021, the entire content of which is incorporated herein by reference for all purposes.

### STATEMENT REGARDING FEDERALLY SPONSORED RESEARCH

This invention was made with government support under Grant no. GM118112, awarded by the National Institutes of Health. The Government has certain rights in the invention.

### TECHNICAL FIELDS OF THE INVENTION

The invention generally relates to novel compounds and methods for protein modification. More particularly, the invention relates to novel compounds and methods for site-specific incorporation of citrulline (Cit) into peptides and proteins in mammalian cells. The invention also relates to modified proteins with site specific citrullination and pharmaceutical compositions and methods of preparation and use thereof.

### BACKGROUND OF THE INVENTION

Citrullination is a post-translational modification (PTM) that involves the hydrolysis of the positively-charged guanidium group on arginine to generate a neutral urea (FIG. 1a). Citrullination plays crucial roles in many physiological processes, including the epigenetic regulation of gene transcription, Neutrophil Extracellular Trap (NET)-formation or NETosis, and maintaining pluripotency. Citrullination is catalyzed by the Protein Arginine Deiminases (PADs) (FIG. 1a), a group of four catalytically active cysteine hydrolases (PAD1-4). PADs are Ca²⁺-dependent enzymes and the presence of calcium increases PAD activity by >10,000-fold. Calcium-binding leads to dramatic conformational rearrangements, particularly of the nucleophilic cysteine (C645 in PAD1, 4; C647 in PAD2; C646 in PAD3) to form a catalytically competent active site. (Fuhrmann, et al. 2015 Chem. Rev. 115, 5413-5461; Brinkmann, et al. 2004 Science 303, 1532-1535; Christophorou, et al. 2014 Nature 507, 104-108; Cuthbert, et al. 2004 Cell 118, 545-553; Kenny, et al. 2017 Elife 6; Tanikawa, et al. 2012 Nat. Commun. 3, 676; Zhang, et al. 2012 Proc. Natl. Acad. Sci. USA 109, 13331-13336; Mondal, et al. 2019 Acc. Chem. Res. 52, 818-832; Slade, et al. 2015 ACS Chem. Biol. 10, 1043-1053.)

Aberrant protein citrullination is a hallmark of multiple autoimmune disorders, including rheumatoid arthritis (RA), multiple sclerosis (MS), ulcerative colitis (UC) and lupus, as well as several neurodegenerative diseases and cancer. Of note, multiple pan- and isozyme-selective PAD inhibitors are known and these inhibitors show efficacy in animal models of RA, UC, MS, lupus and sepsis. The contribution of protein hypercitrullination to the pathology of various diseases has been further established using the phenylglyoxal (PG)-based citrulline-specific probes, Rhodamine-PG (Rh-PG) and Biotin-PG. For example, Rh-PG enabled visualization of extensive citrullination of serum proteins and a marked decrease upon treatment with pan-PAD inhibitor, Cl-amidine, in a mouse model of UC. Using Biotin-PG and a chemoproteomic platform, also identified were various classes of novel citrullinated proteins, including serine protease inhibitors (SERPINs), serine proteases, transport proteins and complement system components along with known citrullinated proteins (e.g., vimentin, enolase, keratin and fibrin) in the serum, synovial fluid and synovial tissue of RA patients. Although the list of citrullinated proteins is ever expanding, the effect of citrullination on the structure and activity of a given protein remains poorly understood. (Fuhrmann, et al. 2015 Chem. Rev. 115, 5413-5461; Mondal, et al. 2019 Acc. Chem. Res. 52, 818-832; Mondal, et al. 2019 Angew. Chem. Int. Ed. 58, 12476-12480.; Mondal, et al. 2018 ACS Chem. Biol. 13, 1057-1065; Wu, et al. Inhibition of PAD2 Improves Survival in a Mouse Model of Lethal LPS-Induced Endotoxic Shock. Inflammation (2020; Bicker, et al. 2012 J. Am. Chem. Soc. 134, 17015-17018; Tilvawala, et al. 2018 Cell Chem. Biol. 25, 691-704 e696.)

Currently, the most commonly used strategy for generating a citrullinated protein involves its treatment with a PAD. However, this leads to citrullination at all sites that are available in vitro, which may not fully recapitulate the situation in vivo. Moreover, the degree of modification at each site is frequently partial, leading to a complex heterogeneous mixture. Clearly, this strategy fails to provide information on the effect of individual citrullination events, underscoring the need for a method to site-specifically incorporate citrulline into proteins.

Although Gln mutations have been used as surrogates for citrulline (Cit), Gln is smaller and does not accurately mimic the H-bonding patterns afforded by Cit. In vitro translation systems or post-translational mutagenesis approaches that have been used to incorporate Cit are limited by their cumbersome nature, the need for specialized equipment, and for the latter approach, the need to incorporate a dehydroalanine at the site of modification, which is itself challenging and generates a mixture of D- and L-stereoisomers. Additionally, these strategies preclude the expression of site-specifically citrullinated proteins in living cells, and therefore, are ineffective for interpreting the downstream implications of this PTM. By contrast genetic code expansion technologies enable the site-specific incorporation of unnatural amino acids (UAAs) into proteins using engineered aminoacyl-tRNA synthetase (aaRS)-tRNA pairs. This technology has been used to genetically encode many important PTMs, enabling the expression of homogeneously modified protein at desired sites in living cells. However, genetically encoding Cit using this technology has remained elusive so far. (Tilvawala, et al. 2018 Cell Chem. Biol. 25, 691-704 e696; Clancy, et al. 2017 ACS Chem. Biol. 12, 1691-1702; Nemmara, et al. 2018 ACS Chem. Biol. 13, 2663-2672; Slack, et al. 2011 Biochemistry 50, 3997-4010; Akahoshi, et al. 2011 Biochem Biophys Res Commun 414, 625-630; Wright, et al. 2016 Science 4, 354, 6312; Chin 2017 Nature 550, 53-60; Dumas, et al. 2015 Chem. Sci. 6, 50-69; Italia, et al. 2017 Nat. Chem. Biol. 13, 446-450; Italia, et al. 2017 Biochem. Soc. Trans. 45, 555-562; Young, et al. 2018 ACS Chem. Biol. 13, 854-870; Groff, et al. 2010 Chembiochem 11, 1066-1068; Italia, et al. 2020 Nat. Chem. Biol. 16, 379-382; Luo, et al. 2017 Nat. Chem. Biol. 13, 845-849; Neumann, et al. 2008 Nat. Chem. Biol. 4, 232-234.)

An ongoing need exists for novel methods for efficient incorporation of Cit into proteins in mammalian cells.

### SUMMARY OF THE INVENTION

The invention is based in part on the unexpected discovery of a novel and improved strategy for facile the site-specific incorporation of citrulline into proteins in mammalian cells. In particular, the disclosed invention exploits an engineered E. coli-derived leucyl tRNA synthetase-tRNA pair that incorporates a photocaged-citrulline (e.g., SM60) into proteins in response to a nonsense codon. Subsequently, SM60 is readily converted to Cit with light in vitro and in living cells. This pair, in response to a nonsense codon (UAG), charges a photocaged-citrulline, SM60 (FIG. 1b and FIG. 1c), into proteins expressed in HEK293T or EXPI293F cells. Subsequently, the photocage is removed with 365 nm UV to generate Cit in vitro or in living cells (FIG. 1c). To demonstrate proof-of-concept, citrulline (Cit) was incorporated at two well-known autocitrullination sites, R372 and R374, in Protein Arginine Deiminase 4 (PAD4) and elucidated how these modifications impact enzyme activity. The results showed that the R372Cit and R374Cit mutants were 181- and 9-fold less active than the wild-type enzyme.

In one aspect, the invention generally relates to an unnatural citrulline analog having the structural formula (I):

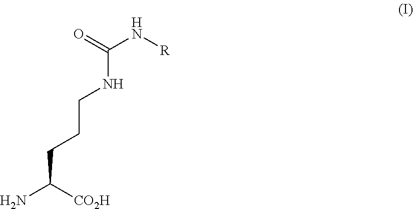

wherein R is a photoreleasable group.

In another aspect, the invention generally relates to a peptide comprising an unnatural citrulline analog disclosed herein.

In yet another aspect, the invention generally relates to a method for treating a disease comprising administering to a patient in need thereof the peptide comprising an unnatural citrulline analog disclosed herein.

In yet another aspect, the invention generally relates to a pharmaceutical composition comprising a peptide comprising an unnatural citrulline analog disclosed herein and a pharmaceutically acceptable excipient, carrier or diluent.

In yet another aspect, the invention generally relates to a method for treating a disease comprising administering to a patient in need thereof the pharmaceutical composition disclosed herein.

In yet another aspect, the invention generally relates to a method for site-specific citrullination in a synthetic peptide. The method comprises: incorporating an unnatural citrulline analog disclosed herein at one or more positions in the synthetic peptide where citrullination is desired; and photochemically converting the unnatural citrulline analog to citrulline.

### BRIEF DESCRIPTION OF THE DRAWINGS

FIG. 1. SM60, a photocaged-citrulline and its conversion to citrulline with 365 nm UV light. a, Conversion of peptidyl-arginine to peptidyl-citrulline by the PADs. b, Chemical structures of SM60 and SM70. c, Schematic representation of the incorporation of SM60 into proteins by an engineered LeuRS-tRNA^{Leu} pair and subsequent conversion to citrulline. d, Decaging of SM60 to citrulline with 365 nm UV irradiation. Left and right panels indicate the HPLC and ion chromatograms, showing the disappearance of SM60 and the formation of citrulline (Cit), respectively, with increasing UV exposure. Quantitative analyses of decaging and Cit formation are shown in the insets. Assay mixture: 1 mM SM60, 2 mM DTT, Phosphate-buffered saline pH 7.4.

FIG. 2. Site-specific incorporation of SM60 in EGFP and subsequent conversion to citrulline in HEK293T cells. a, EGFP-39-TAG reporter expression by EcLeuRS-tRNA_{CUA}^{EcLeu} pair in HEK293T cells in the presence of SM60 indicated by the fluorescence of EGFP. b, Quantification of EGFP-39-TAG reporter expression efficiency in the presence of increasing concentration of SM60. c, Coomassie stain of purified EGFP containing SM60. Full gel is given in FIG. 11. d, Deconvoluted mass spectrum of EGFP before and after 365 nm UV irradiation (1 min), indicating the presence of SM60 and citrulline, respectively, at 39 position. Non-deconvoluted spectra are given in FIG. 12 and FIG. 13.

FIG. 3. Autocitrullination of PAD4, incorporation of citrulline in PAD4 and implications thereof. a, Chemical structure of Rh-PG and the fluorescence labeling of autocitrullinated PAD4. The bands at 0 min in the presence of calcium and at all the time-points in the absence of calcium correspond to the basal levels of autocitrullination during the expression and purification of PAD4 from E. coli. b, Expression of R372Cit PAD4 in the presence of engineered release factor, eRF1-E55D and SM60 in HEK293T cells, indicating the essential role of eRF1-E55D for efficient TAG-suppression. c, Coomassie stains for WT, R372Cit and R374Cit PAD4, indicating their purity. d, Michaelis-Menten kinetics for WT, R372Cit and R374Cit PAD4. e, Western blot analysis of histone H3 citrullination in live HEK293T cells by WT and R374Cit PAD4. The normalization procedure is given in the supporting information. f, Thermal shift profiles of WT and R374Cit PAD4. Western blot images are given in supplementary FIG. 21. The table indicates the melting temperatures (Tₘ). g, Effect of autocitrullination on the enzymatic activity of PAD4.

FIG. 4. Autocitrullination sites in PAD4. a, Heat map representing the time-dependent change in peptides containing arginine or citrulline at the indicated positions (autocitrullination sites). Ca²⁺-untreated samples are negative controls. b, Time-dependent autocitrullination at the major sites. R218 site could not be shown because of disorder in that region (PDB: 1WDA). The increase in autocitrullination at these sites follows the same color code as in panel a.

FIG. 5. ¹H (A) and ¹³C (B) NMR of SM60 in DMSO-d₆.

FIG. 6. ESI Mass spectrum of SM60 (A) and SM70 (B).

FIG. 7. ESI mass spectrum for the formation of citrulline from SM60 upon UV exposure. Citrulline appears at ˜0.6 min in the ion chromatogram (FIG. 1D). While the peaks for citrulline are absent before UV exposure (A), they appear upon UV exposure and reach a maximum after 5 min treatment. The representative mass spectrum after 5 min exposure is shown in panel B. (C) ESI mass spectrum of a standard sample of citrulline. ESI-MS (m/z) calculated for C₆H₁₃N₃O₃ [M+H]⁺: 176.10, found 176.20; [M+Na]⁺: 198.09, found 198.20.

FIG. 8. ¹H NMR spectrum of SM60 in D₂O after UV (365 nm) exposure for 1, 2, 3, 4 and 5 min. The disappearance of the benzylic ‘a’ protons at 4.5 ppm (as seen in the spectrum denoted by 0 min) with increasing UV exposure indicates the complete removal of the o-nitrobenzyl photocage. Furthermore, an upfield shift was also observed for the ‘b’ protons upon the formation of citrulline. The multiplets at 3.6-3.7 and 2.6-2.7 ppm are due to DTT used in the assay. Assay condition: 1 mM SM60, 2 mM DTT, D₂O.

FIG. 9. (A) Photodecaging of Fmoc-SM60 to produce Fmoc-Cit. (B) HPLC chromatograms indicating the disappearance of Fmoc-SM60 and the production of Fmoc-Cit with increasing UV exposure. These chromatograms also indicate the quantitative conversion of Fmoc-SM60 to Fmoc-Cit. Assay mixture: 0.5 mM Fmoc-SM60, 2 mM DTT, Phosphate-buffered saline pH 7.4. ESI-Mass spectra of Fmoc-SM60 (C) and Fmoc-Cit (D).

FIG. 10. (A) Decaging of SM70, a PAD4-derived peptide (residues 363-372 with SM60 at the 372 position. Prolines at 365 and 371 positions were replaced with W and A, respectively, and a lysine residue was added to the C-terminus for ease of synthesis.). (B) HPLC chromatogram of SM70 before and after 5 min of 365 nm irradiation. The low peak intensity of the decaged product is explained by the lack of an aromatic group and its extremely low solubility in aqueous buffer. Even after performing the reaction in 1:1 acetonitrile/PBS, the solution became turbid upon the formation of the decaged peptide. Assay condition: 200 μM SM70, 2 mM DTT, 1:1 acetonitrile/PBS (pH 7.4). (C) ESI mass spectrum of the citrulline-containing peptide (decaged product).

FIG. 11. Coomassie stain of purified EGFP containing SM60 at 39 position.

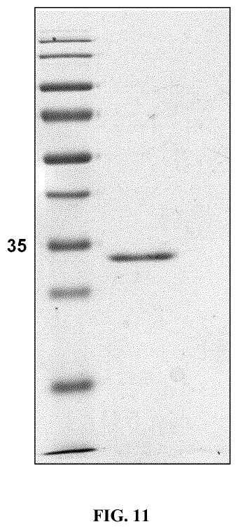

FIG. 12. ESI mass spectrum (A) and deconvoluted spectra (B) of EGFP containing SM60 at 39 position.

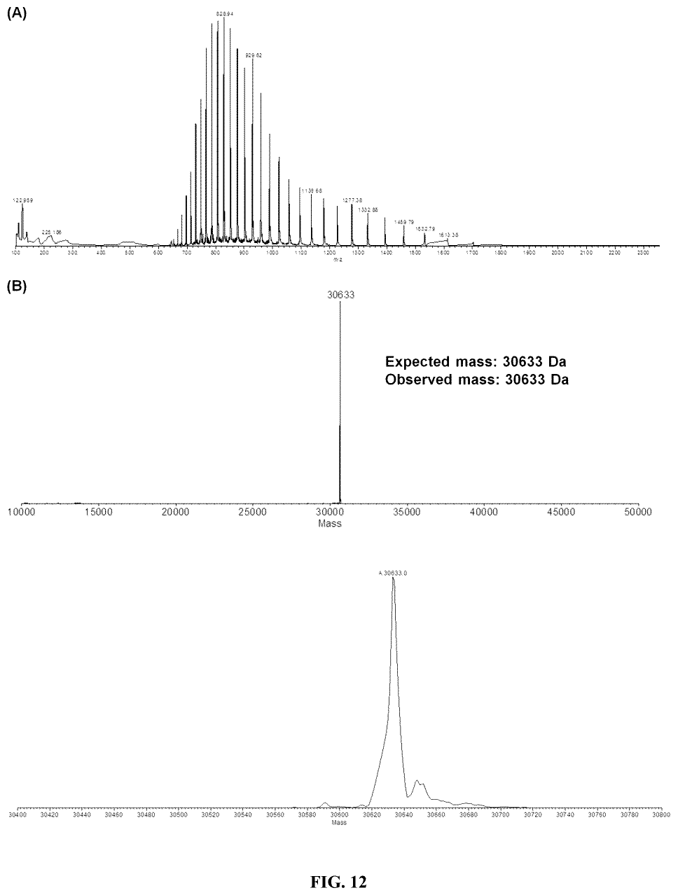

FIG. 13. ESI mass spectrum (A) and deconvoluted spectra (B) of EGFP containing Cit at 39 position.

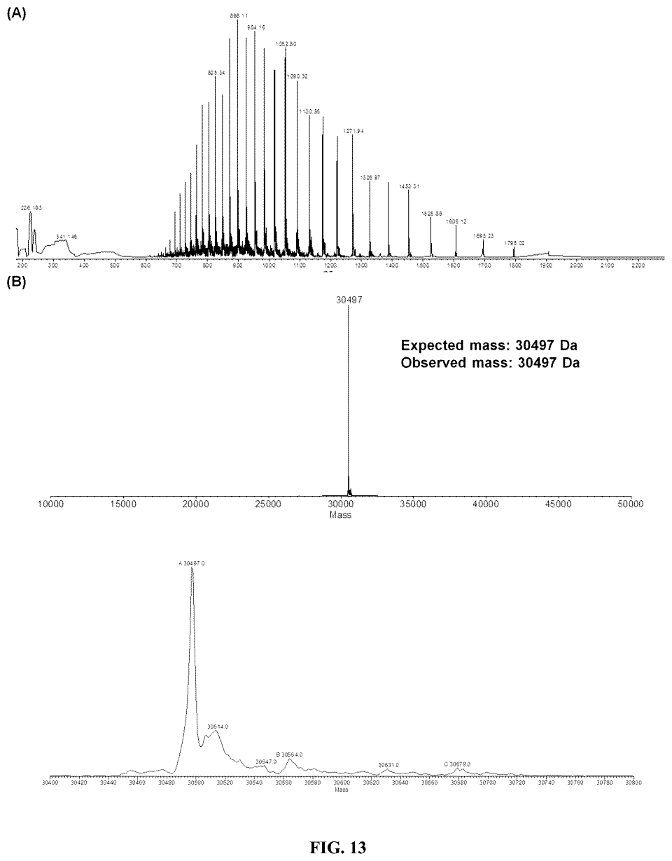

FIG. 14. (A) Viability of HEK293T cells treated with increasing concentrations of SM60, and a combination of SM60 and 365 nm UV. The dotted line indicates the concentration (1 mM) used for citrulline incorporation in GFP and PAD4. (B) Viability of HEK293T cells overexpressing WT and R374Cit PAD4 before and after photodecaging. These results indicate that PAD4 expression as well as light treatment does not cause cell death. Various concentrations of SM60 were used to induce increasing amounts of R374Cit mutant expression.

FIG. 15. Sites of PAD4 autocitrullination (Table 1) (PDB code: 1WDA). R218 site could not be shown because of disorder in that region. While most of these sites are far from the active site and are on the surface of the protein, arginines 372 and 374 are close to the active site. Notably, the distance between these two positively-charged residues is only 3.5 Å, a value that is close to the sum of N . . . N van der Waal's radii (3.1 Å). Electrostatic repulsions between R372 and R374 are delicately balanced by hydrogen bonding interactions with the substrate, BAA and an aspartate, D345. These observations suggest that the citrullination of either of these residues will likely perturb the delicate balance and affect enzymatic activity.

FIG. 16. MS2 spectrum of the peptide (³⁶³TLPVVFDSPCitNRGLK³⁷⁷) containing the Cit372 residue. This peptide was generated by digesting the R372Cit mutant PAD4 with Lys-C and Glu-C. A 43 Da neutral loss, characteristic to the citrulline side chain that loses isocyanic acid during fragmentation, from y7-y10, and not from y4-y5 ion confirms citrulline incorporation at the 372 position.

FIG. 17. MS2 spectrum of the peptide (³⁷⁰SPRNCitGLKEFPIK³⁸²) containing the Cit374 residue. This peptide was generated by digesting R374Cit mutant PAD4 with Lys-C and Glu-C. A 43 Da neutral loss, characteristic to the citrulline side chain that loses isocyanic acid during fragmentation, from b5, and not from b4 ion confirms citrulline incorporation at the 374 position.

FIG. 18. (A) Expression of WT and R374Cit PAD4 in EXPI293F cells as monitored by western blot analysis of the lysate using an a-PAD4 antibody. EXPI293F cell lysate (without transfection) served as a negative control. (B) Coomassie stain of purified WT and R374Cit PAD4 from EXPI293F lysate by Ni-NTA affinity chromatography. Wash 1, Wash 2 and Elution buffer contained 50, 75 and 300 mM imidazole, and the elution fraction was dialyzed to remove the imidazole.

FIG. 19. (A) Michaelis-Menten kinetics for the citrullination of BAEE by PAD4_{Mam} (purified from mammalian expression system) and PAD4_{Bac} (purified from bacterial expression system), indicating that both these enzymes have similar steady-state kinetic parameters (Table 3). Since PAD4_{Mam} contains an N-terminal FLAG and a C-terminal His tag, these results also indicate that the presence of these tags do not affect enzymatic activity. (B) Calcium-dependence plots for PAD4_{Mam} and PAD4_{Bac}. These data indicate that regardless of the source of the enzyme, the K_{0.5} (Ca²⁺ concentration for half-maximal activity) values are 0.9 mM. (C) Time-dependent citrulline production by WT, R372Cit and R374Cit PAD4. (D) Effect of autocitrullination on the enzymatic activity of PAD4. The activity loss both in the presence and absence of calcium may be due to the oxidation of the active site cysteine over time.

FIG. 20. (A) Chemical structure of rhodamine-conjugated F-amidine (RFA). (B) RFA-labeling of wild-type (WT) and R374Cit PAD4 in the presence and absence of Ca²⁺. (C) RFA-labeling of EXPI293F cell lysate containing wild-type (WT) and R374Cit PAD4. EXPI293F cell lysate (without transfection) served as a negative control. F and C refer to the fluorograph and coomassie brilliant blue stain, respectively.

FIG. 21. Full western blot images for the thermal shift assays using EXPI293F cell lysate containing wild-type (WT) and R374Cit PAD4. GSK199 and Ca2⁺ were used at 10 μM and 1 mM concentration, respectively. Full-length PAD4 is indicated by a brown triangle in each blot.

FIG. 22. (A) Thermal stability of WT and R374Cit PAD4 at 37° C. over 3 h. EXPI293F cell lysate containing WT PAD4 or R374Cit mutant was used in this assay. Full-length PAD4 is indicated by a brown triangle in each blot. (B) Variation of PAD4 band intensities in panel A over 180 min, indicating that R374Cit mutant is as stable as WT PAD4 at 37° C.

FIG. 23. Schematic representation of quantitative proteomic analysis of time-dependent autocitrullination of PAD4. Samples treated in the absence of calcium served as negative controls. Peptides derived from proteolysis of PAD4 with Lys-C and Glu-C were labeled with isobaric tandem mass tags (TMT) to enable simultaneous quantification of autocitrullination at various sites with increasing time.

### DETAILED DESCRIPTION OF THE INVENTION

The invention provides a novel and improved strategy that enables facile the site-specific incorporation of citrulline (Cit) into proteins in mammalian cells using an E. coli-derived engineered leucyl-tRNA synthetase (EcLeuRS)-tRNAcuAaLeu pair

Disclosed here is the development of a novel technique for the site-specific incorporation of citrulline into proteins in mammalian cells. Central to this technology are a photocaged citrulline (e.g., SM60) and an E. coli-derived engineered leucyl-tRNA synthetase (EcLeuRS)/tRNA_{CUA}^{EcLeu} pair that enables the incorporation of SM60 into proteins in response to a TAG nonsense codon with high fidelity and efficiency. Subsequently, the photocage is removed with 365 nm light to generate citrulline. This technique overcomes several limitations of previous methods used to incorporate citrulline, including in vitro translation, post-translational mutagenesis, and in vivo nonsense suppression by chemically acylated tRNAs. (Akahoshi, et al. 2011 Biochem Biophys Res Commun 414, 625-630; Wright, et al. 2016 Science 4, 354, 6312; Infield, et al. 2018 J. Gen. Physiol. 150, 1017-1024.)

For example, chemically acylated tRNAs are not readily synthesized, cannot be regenerated, and consequently give poor yields. By contrast, the dislcosed approach provides a highly scalable expression platform that can be readily adapted by virtually any lab to site-specifically incorporate Cit into any mammalian protein, and thereby facilitate cellular studies to understand the downstream implications of this PTM.

Specifically, citrulline was incorporated at two known autocitrullination sites, R372 and R374, in PAD4. Kinetic studies indicate that the R374Cit and R372Cit mutants are 9- and 181-fold less active than WT PAD4. Detailed studies indicate that citrullination induces local conformational changes within the active site that leads to slow reaction between C645 and the guanidium group of the substrate, the first step in the catalytic cycle. While these results indicate that citrullination of R372 and R374 would decrease PAD4 activity, it was found that autocitrullination does not impact the enzymatic activity. Quantitative proteomics studies indicate that 212/218 and 484/488/495, and not 372 and 374, are the preferred sites of citrullination. While faster autocitrullination of arginines 212/218 and 484/488/495 is likely due to their residence at the surface of PAD4, upon citrullination, they may expose deeply buried autocitrullination sites by conformational changes. Efforts are currently under way to elucidate the effect of citrullination at these major sites, particularly the 484/488/495 residues because they are present at the interface of the head-to-tail PAD4 dimer that is known to alter enzymatic activity.

Since it is well established that citrullination is critical for many physiological processes, as well as in disease pathology, this new method will provide a direct and accessible approach to understand the biology of this PTM at the molecular level. For example, histone H3 citrullination at R26 leads to the transcriptional activation of more than 200 genes in estrogen receptor-positive breast cancer cells and inhibits the methylation of the neighboring 1(27 residue by 30,000-fold. However, the mechanism of negative crosstalk between these two PTMs remains poorly understood. Additionally, it was recently showed that serine protease inhibitors (SERPINs), nicotinamide N-methyl transferase (NNMT), and pyruvate kinase isoform M2 (PKM2) are citrullinated in patients suffering from rheumatoid arthritis. Notably, the citrullination of the SERPINs and NNMT dramatically abolishes their enzymatic activity, while citrullinated PKM2 exhibits 2-3-fold higher activity than the WT enzyme. However, the underlying reasons behind such biochemical phenomenon are unclear. Finally, citrullination has been reported to impact neutrophil extracellular trap formation, pluripotency, and efficient elongation by RNA PolII but, again, the underlying mechanisms remain unclear. With the disclosed technology, it is now possible to incorporate citrulline on demand and mechanistically address how this PTM impacts these fundamental biological processes and pathways.

In one aspect, the invention generally relates to an unnatural citrulline analog having the structural formula (I):

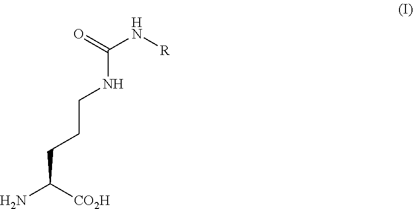

wherein R is a photoreleasable group.

By the term “photoreleasable” it is meant a bond capable of being broken resulting via a photochemical reaction by photons (e.g., a photolytic reaction). Thus, a photoreleasable group refers to a group within a compound that departs from the rest of the molecule as a result of a photolytic reaction.

In certain embodiments, R is:

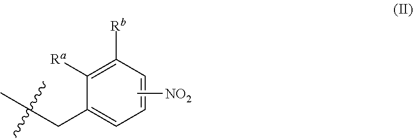

wherein each of R^{a} and R^{b} is selected from H or OR^{c}, wherein R^{c} is a C₁-C₆ alkyl group.

In certain embodiments, R is:

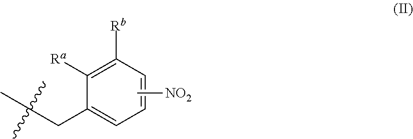

wherein each of R^{a} and R^{b} is selected from H or OR^{c}, wherein R^{c} is a C₁-C₆ alkyl group.

In certain embodiments, R is:

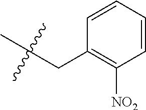 [SMILES: *Cc1ccccc1[N+](=O)[O-]]

In certain embodiments, R is:

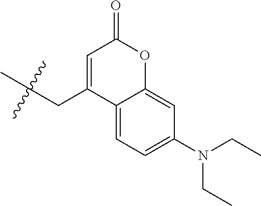 [SMILES: *Cc1cc(=O)oc2cc(N(CC)CC)ccc12]

In certain embodiments, R is:

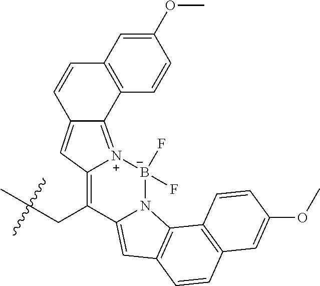 [SMILES: *CC1=C2C=c3ccc4cc(OC)ccc4c3=[N+]2[B-](F)(F)n2c1cc1ccc3cc(OC)ccc3c12]

In another aspect, the invention generally relates to a peptide comprising an unnatural citrulline analog disclosed herein.

In yet another aspect, the invention generally relates to a method for treating a disease comprising administering to a patient in need thereof the peptide comprising an unnatural citrulline analog disclosed herein.

In yet another aspect, the invention generally relates to a pharmaceutical composition comprising a peptide comprising an unnatural citrulline analog disclosed herein and a pharmaceutically acceptable excipient, carrier or diluent.

In yet another aspect, the invention generally relates to a method for treating a disease comprising administering to a patient in need thereof the pharmaceutical composition disclosed herein.

In yet another aspect, the invention generally relates to a method for site-specific citrullination in a synthetic peptide. The method comprises: incorporating an unnatural citrulline analog disclosed herein at one or more positions in the synthetic peptide where citrullination is desired; and photochemically converting the unnatural citrulline analog to citrulline.

In certain embodiments, incorporating an unnatural citrulline analog is achieved by using an E. coli-derived engineered leucyl-tRNA synthetase (EcLeuRS)/tRNA_{CUA}^{EcLeu} pair

In certain embodiments, photochemical conversion of the unnatural citrulline analog to citrulline comprises directing a UV irradiation at the synthetic peptide.

In certain embodiments, the UV-Visible irradiation comprises wavelengths in the range of about 250 nm to about 700 nm (e.g., about 365 nm to about 700 nm). In certain embodiments, the UV irradiation comprises the wavelength of 365 nm.

In certain embodiments, the photochemical conversion results in greater than 90% (e.g., >95%, >98%) conversion of the unnatural citrulline analog to citrulline.

As used herein, the terms “protein” and “peptide” are used interchangeably to refer to a polymer of amino acid residues, and are not limited to a minimum length. Thus, peptides, oligopeptides, polypeptides, dimers, multimers, and the like, are included within the definition. Both full-length proteins and fragments thereof are encompassed by the definition. The terms also include post-expression modifications of the protein, for example, glycosylation, acetylation, phosphorylation, and the like. Furthermore, a peptide may refer to a protein which includes modifications, such as deletions, additions, and substitutions (generally conservative in nature), to the native sequence, as long as the protein maintains the desired activity. These modifications may be deliberate or may be accidental. Amino acids can be referred to herein by either their commonly known three letter symbols or by the one-letter symbols recommended by the IUPAC-IUB Biochemical Nomenclature Commission.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Solvates and polymorphs of the compounds of the invention are also contemplated herein. Solvates of the compounds of the present invention include, for example, hydrates.

Definitions of specific functional groups and chemical terms are described in more detail below. When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure. Solvates and polymorphs of the compounds of the invention are also contemplated herein. Solvates of the compounds of the present invention include, for example, hydrates.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

The following examples are meant to be illustrative of the practice of the invention and not limiting in any way.

### EXAMPLES

The below Examples describe certain exemplary embodiments of compounds prepared according to the disclosed invention. It will be appreciated that the following general methods, and other methods known to one of ordinary skill in the art, can be applied to compounds and subclasses and species thereof, as disclosed herein.

### SM60: A Photocaged-Citrulline

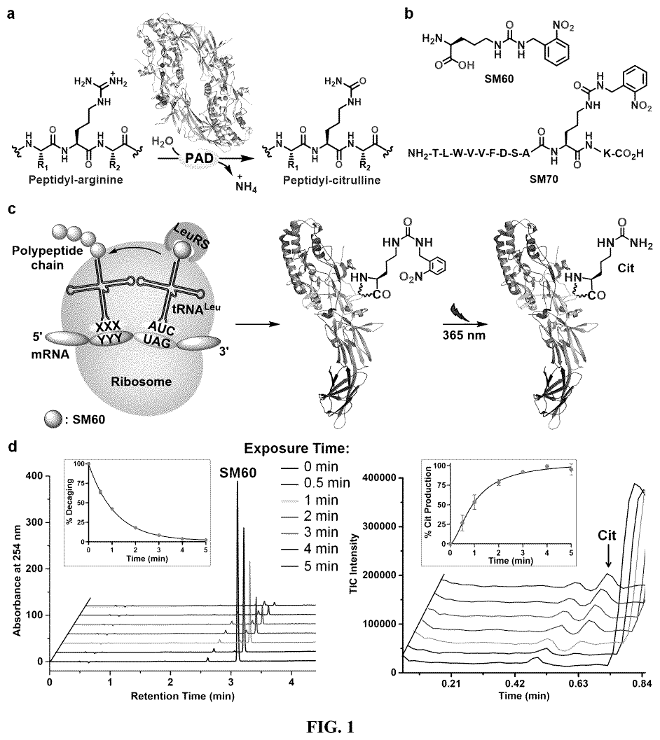

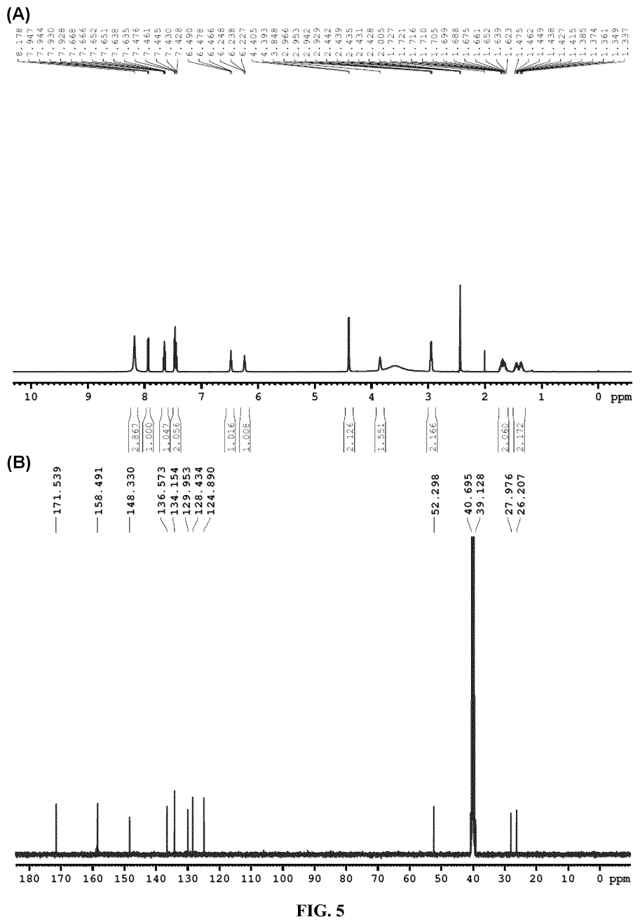

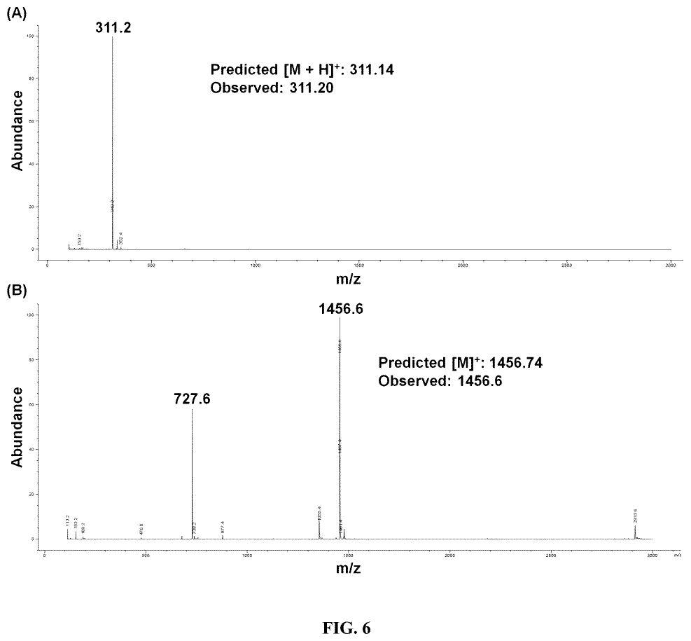

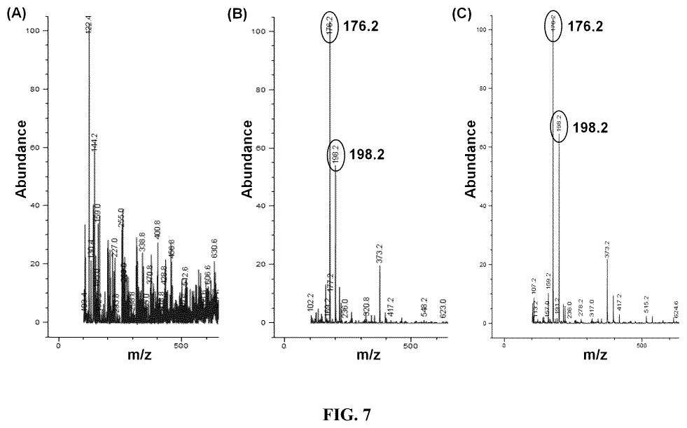

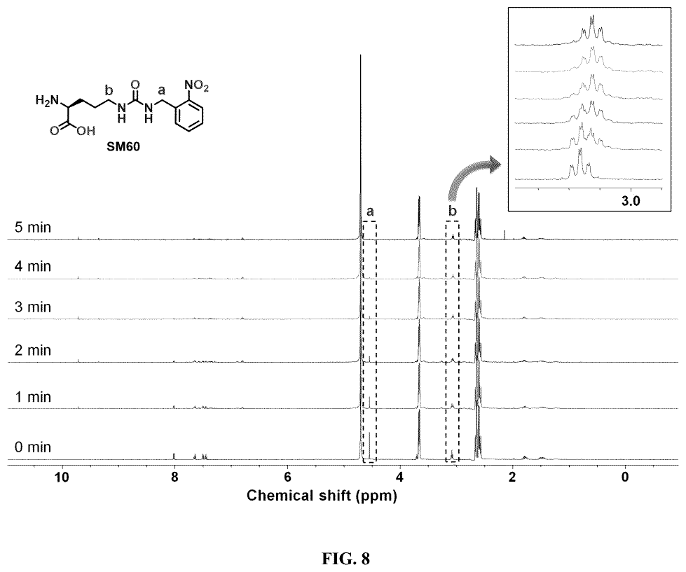

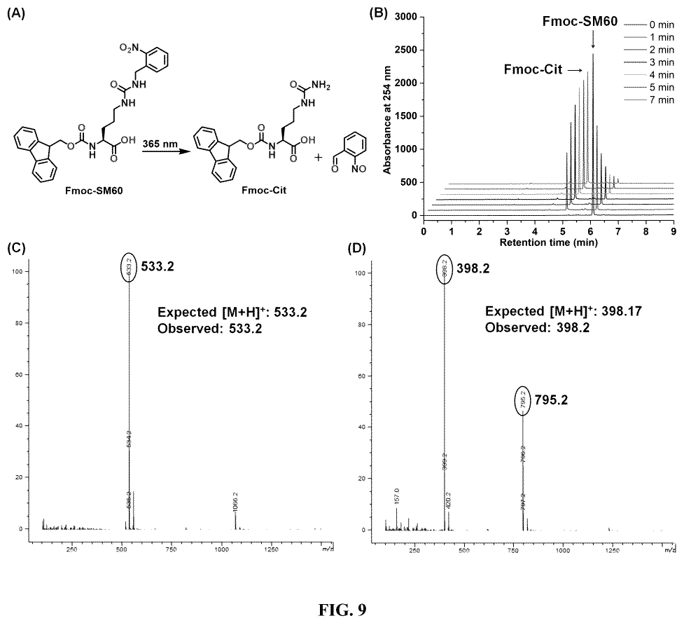

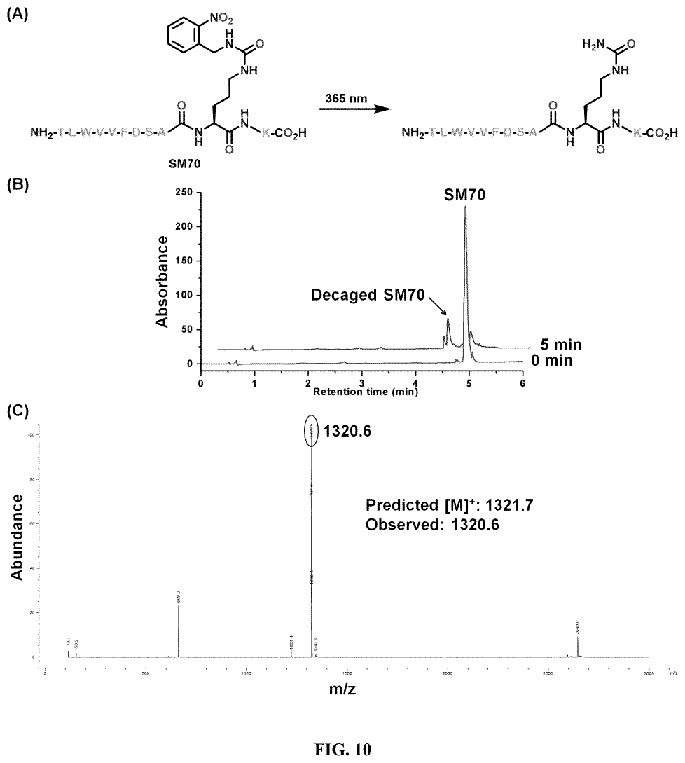

Envisioning that it may be challenging to develop an engineered aaRS that would selectively charge Cit, while discriminating against a nearly isostructural arginine, a caging strategy was developed. In particular, a photocaged-citrulline (SM60, comprising an o-nitrobenzyl photocage on the Cit side chain) was designed, which is structurally distinct from the 20 canonical amino acids but can be efficiently converted to Cit post-translationally (FIGS. 1b, 1d). SM60 was synthesized over two steps from L-ornithine, and was characterized by ¹H, ¹³C NMR spectroscopy and mass spectrometry (FIG. 5 and FIG. 6a). Using LC-MS, it was found that SM60 can be quantitatively converted to Cit in phosphate-buffered saline (PBS) supplemented with dithiothreitol (DTT) using 365 nm UV radiation for 5 min (FIG. 1d and FIG. 7). Quantitative conversion was further supported by ¹H NMR analysis, which shows the rapid disappearance of the benzylic protons of SM60 at 4.5 ppm with increasing UV exposure and by the photodecaging of Fmoc-SM60 to Fmoc-Cit (FIG. 8 and FIG. 9). To further investigate the feasibility of decaging SM60 on proteins, a SM60-containing peptide, SM70 (FIG. 1b and FIG. 6b) was synthesized that contains residues 363-372 of PAD4 with SM60 at the 372 position, a known autocitrullination site. Gratifyingly, SM70 also undergoes photodecaging to form the citrulline-containing peptide (FIG. 10), indicating that SM60 can be decaged in the presence of other amino acids.

### Genetically Encoding SM60 in Eukaryotes

Four different aaRS/tRNA pairs have been successfully engineered for incorporating UAAs in eukaryotic cells: bacteria-derived tyrosyl, tryptophanyl, and leucyl pairs and the archaea-derived pyrrolysyl pair. (Dumas, et al. 2015 Chem. Sci. 6, 50-69; Italia, et al. 2017 Nat. Chem. Biol. 13, 446-450; Italia, et al. 2017 Biochem. Soc. Trans. 45, 555-562; Young, et al. 2018 ACS Chem. Biol. 13, 854-870; Chin, et al. 2003 Science 301, 964-967; Wu, et al. 2004 J. Am. Chem. Soc. 126, 14306-14307; Zheng, et al. 2018 Biochemistry 57, 441-445.)

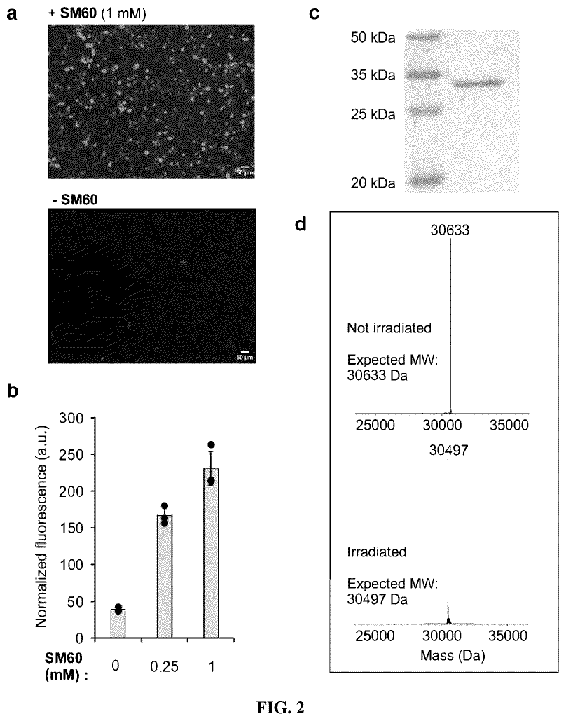

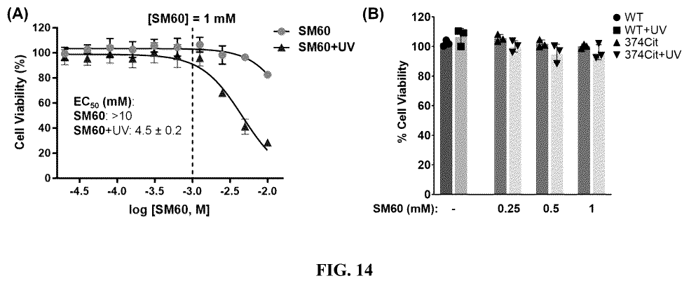

The first two pairs are restricted to structural analogs of phenylalanine and tryptophan, respectively, precluding their use to genetically encode SM60. However, both the archaeal pyrrolysyl (PylRS/tRNA^{Pyl}) and E. coli leucyl (EcLeuRS-tRNA_{CUA}^{EcLeu}) pairs have been engineered to charge UAAs structurally similar to SM60. Engineered aaRSs often exhibit substrate polyspecificity, i.e. the ability to use several structurally analogous UAAs, while discriminating against the canonical amino acids. This property has provided a facile route to rapidly expand the repertoire of genetically encoded UAAs without having to engineer new aaRS mutants for each distinct substrate. To explore if such a polyspecific aaRS can accept SM60 as a substrate, several existing PylRS and EcLeuRS mutants were screened using an EGFP-39-TAG expression assay in HEK293T cells in the presence of their cognate amber suppressor tRNA. This screen identified an EcLeuRS mutant (M40I, Y499I, Y527A and H529G in the active site, and T252A in the editing domain)³¹ which enabled robust expression of the fluorescence reporter only when SM60 was supplemented in the medium (FIG. 2a and FIG. 2b). Purification of the resulting full-length EGFP using a C-terminal polyhistidine tag, followed by mass spectrometry, showed a mass consistent with the successful incorporation of SM60 (FIG. 2c and FIG. 2d). Furthermore, UV irradiation of cells expressing EGFP-39-SM60 before lysis followed by protein purification and MS analysis afforded a single protein mass consistent with the complete deprotection and incorporation of citrulline at position 39 of EGFP (FIG. 2d). Notably, SM60 is completely nontoxic (EC₅₀>10 mM) in HEK293T cells at the concentration used for nonsense suppression, i.e. 1 mM (FIG. 14A). Furthermore, a combination of SM60 and 365 nm UV exhibits an EC₅₀ of 4.5±0.2 mM for the inhibition of cell proliferation, indicating that the products of the photodecaging reaction (nitrosobenzaldehyde and citrulline) have negligible cytotoxicity at the working concentration (FIG. 14A).

### Site-Specific Incorporation of Citrulline in PAD4

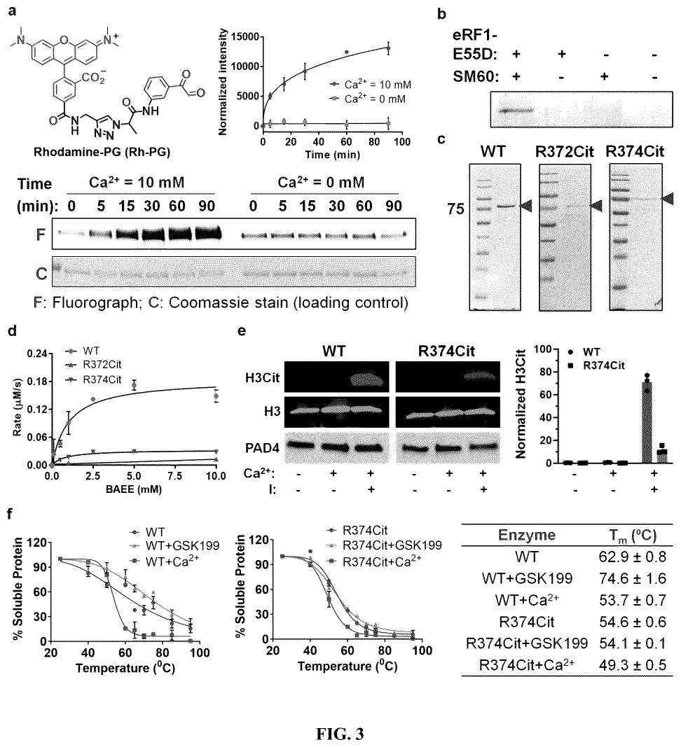

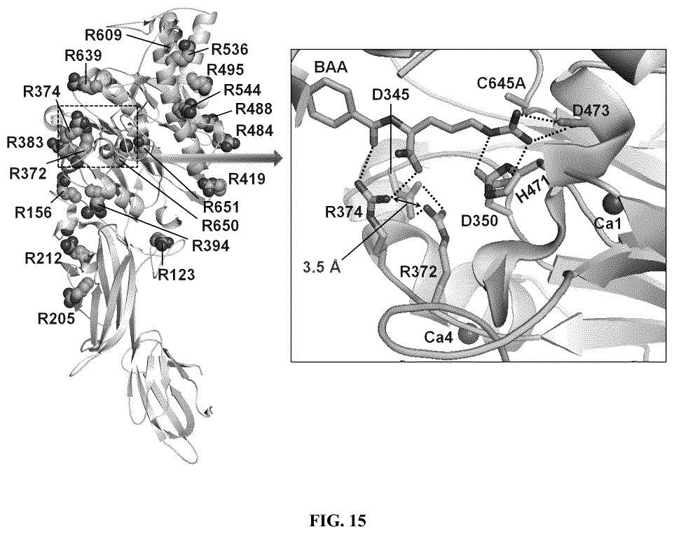

Having established the ability to site-specifically incorporate Cit into EGFP, this technology was exploited to address the effect of autocitrullination on PAD4 activity. These studies were conducted because the effect of autocitrullination on PAD4 activity has been debated. While Andrade et al. reported that autocitrullination negatively impacts PAD4 activity, it was shown that autocitrullination has little to no impact on PAD4 activity. Using a citrulline-specific fluorescent probe Rh-PG, it was confirmed that PAD4 autocitrullinates in the presence of Ca⁺² in a time-dependent manner (FIG. 3a). Several autocitrullination sites in PAD4 (Table 1 and FIG. 15) have previously mapped. (Bicker, et al. 2012 J. Am. Chem. Soc. 134, 17015-17018; Slack, et al. 2011 Biochemistry 50, 3997-4010; Andrade, et al. 2010 Arthritis Rheum. 62, 1630-1640.)

TABLE 1
Sites of autocitrullination in PAD4 and peptides from which they are detected by
tandem mass spectrometry.

| Site | Peptide | Protease Used | Reference |
|---|---|---|---|
| R123 | ISLCADITRTGK | Glu-C/Lys-C | This study |
| | ALLYLTAVEISLCADITRTGK | Trypsin | 1ª |
| | ALLYLTAVEISLCADITR | Trypsin | 1^{b} |
| R156 | TWTWGPCGQGAILLVNCDR | Trypsin | 1^{b} |
| R205 | DFFTNHTLVLHVAR | Trypsin | 1^{b} |
| | DFFTNHTLVLHVARSEMDK | Trypsin | 2 |
| R212/218 | MDKVRVFQATRGK | Glu-C/Lys-C | This study |
| R218 | VRVFQATRGK | Trypsin | 2 |
| | VFQATRGK | Trypsin | 2 |
| R372 | TLPVVFDSPRNRGLK | Glu-C/Lys-C | This study |
| R374 | TLPVVFDSPRNRGLKE | Glu-C/Lys-C | This study |
| R372/374 | TLPVVFDSPRNRGLKE | Glu-C/Lys-C | This study |
| | TLPVVFDSPRNRGLK | Trypsin | 2 |
| R383 | RVMGPDFGYVTR | Trypsin | 2 |
| R394 | RVMGPDFGYVTRGPQTGGISGLDSFGNLE | Glu-C/Lys-C | This study |
| | VMGPDFGYVTR | Trypsin | 2 |
| R419 | VSPPVTVRGK | Glu-C/Lys-C | This study |
| | GPQTGGISGLDSFGNLEVSPPVTVR | Trypsin | 1^{c} |
| R484 | FLSFVPAPDRK | Glu-C/Lys-C | This study |
| | LYSDWLSVGHVDEFLSFVPAPDR | Trypsin | 1^{b} |
| R484/488/495 | FLSFVPAPDRKGFRLLLASPRSCYK | Glu-C/Lys-C | This study |
| R488/R495 | GFRLLLASPRSCYK | Glu-C/Lys-C | This study |
| R495 | GFRLLLASPRSCYK | Glu-C/Lys-C | This study |
| | LLLASPRSCYK | Trypsin | 2 |
| R536 | NILSNKTLRE | Glu-C/Lys-C | This study |
| R544 | HNSFVERCIDWNRE | Glu-C/Lys-C | This study |
| R536/R544 | TLREHNSFVER | Trypsin | 2 |
| R609 | HLGIPKPFGPVINGR | Trypsin | 1ª |
| R639 | VCSLLEPLGLQCTFINDFFTYHIR | Trypsin | 1^{b} |
| R650/651 | VHCGTNVÆRKPFSFK | Glu-C/Lys-C | This study |

^{a}Detected from endogenous PAD4 in HL-60 cells.
^{b}Detected from recombinant PAD4 reconstituted in ¹⁸O labeled water.
^{c}Detected from recombinant PAD4 reconstituted in normal water.

While most of these sites are on the surface, the frequently observed R372 and R374 sites are present in the active site. Notably, the guanidinium groups on these two residues are only 3.5 Å from each other and the expected electrostatic repulsions are delicately balanced by H-bonding and salt-bridge interactions with D345. Moreover, R374 forms two H-bonds with the small molecule substrate, BAA. (Arita, et al. 2004 Nat. Struct. Mol. Biol. 11, 777-783.)

To evaluate whether citrullination at these sites would significantly impact enzyme activity, Cit was incorporated at positions 372 and 374 in PAD4. Wild-type (WT) PAD4 and the 372 and 374 TAG mutants were separately cloned into a pAcBac3 plasmid, which also encodes the mutant EcLeuRS and 8 copies of the tRNA_{CUA}^{EcLeu}. (Zheng, et al. 2018 Biochemistry 57, 441-445; Zheng, et al. 2017 Chem. Sci. 8, 7211-7217.)

Subsequently, these plasmids were transfected separately into HEK293T cells, and the PAD4 protein or its mutants (after irradiation to remove the photocage), were purified using a C-terminal polyhistidine tag. While WT PAD4 expression was robust (10 μg/10⁷ cells), yields for the mutants were very low, indicating poor suppression efficiency at these sites. The Chin group has recently reported a mutant eukaryotic release factor (eRF1 E55D) that can enhance TAG-suppression efficiency in mammalian cells upon overexpression. (Schmied, et al. 2014 J. Am. Chem. Soc. 136, 15577-15583.)

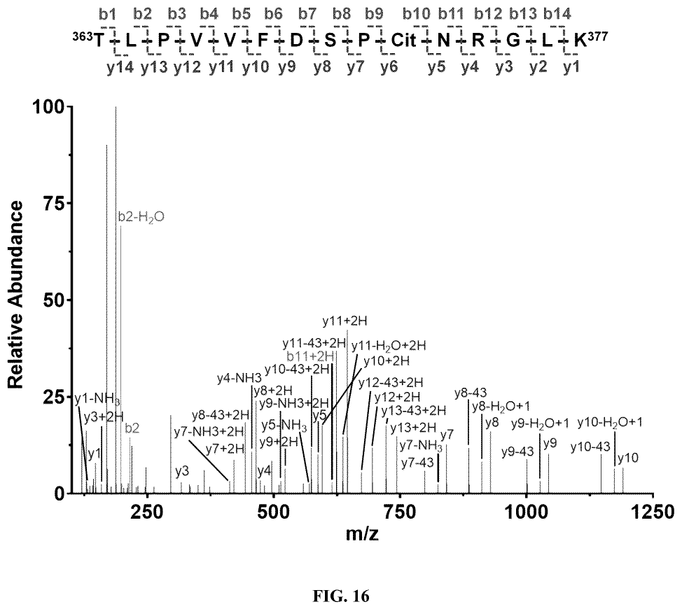

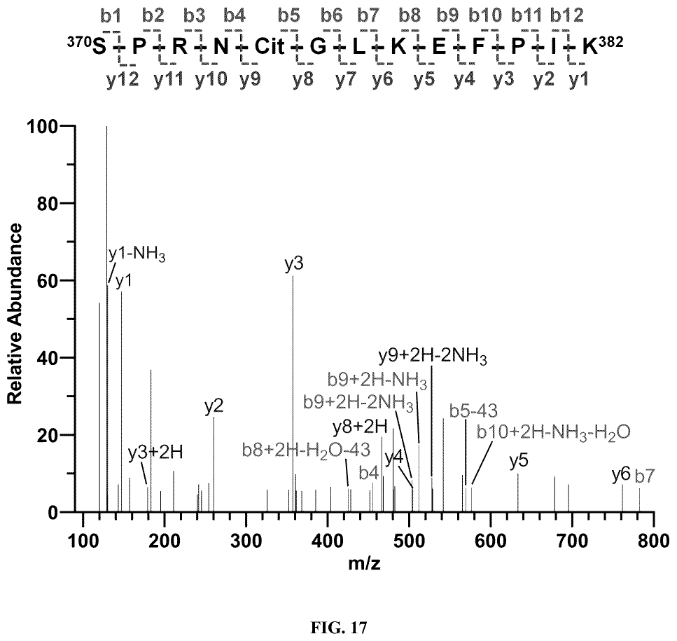

To explore if this strategy can overcome the low suppression efficiency at the target sites in PAD4, eRF1-E55D mutant was cloned under a CMV promoter in a pIDTSMART vector. Indeed, co-transfection of this plasmid significantly improved the efficiency of nonsense suppression and enabled the purification of the desired mutants (2-4 μg/10⁷ cells, FIG. 3b and FIG. 3c). Since the +0.98 Da mass change upon citrullination is difficult to detect by intact MS analysis, the incorporation of citrulline at the desired position was confirmed by LC-MS/MS analysis of the peptides resulting from Lys-C/Glu-C digestion of PAD4 (FIG. 16, FIG. 17, Table 2 and Table 3).

TABLE 2
Ions Detected from LC-MS/MS Analysis of R372Cit PAD4 digested with Lys-C and
Glu-C.

| | | Observed | Actual |
|---|---|---|---|
| Peptide sequence | Charge | Mass | Mass |
| ⁶¹KSTGSSTWPLDPGVE⁷⁵ | 2 | 780.88 | 1,559.75 |
| ⁶²STGSSTWPLDPGVE⁷⁵ | 2 | 716.83 | 1,431.65 |
| ⁹²VQISYYGPKTPPVK¹⁰⁵ | 3 | 526.30 | 1,575.87 |
| ¹¹⁵ISLCADITRTGK¹²⁶ | 3 | 445.57 | 1,333.70* |
| ¹⁸⁰MSLMTLSTKTPK¹⁹¹ | 3 | 446.58 | 1,336.71 |
| ¹⁹²DFFTNHTLVLHVARSE²⁰⁷ | 3 | 629.32 | 1884.95 |
| ¹⁹²DFFTNHTLVLHVARSE²⁰⁷ | 4 | 472.24 | 1,884.95 |
| ²⁸⁸SVVFRVAPWIMTPNTQPPQE³⁰⁷ | 2 | 1149.09 | 2,296.16 |
| | 3 | 766.40 | 2,296.16 |
| | 3 | 771.73 | 2312.16^{†} |
| ³¹⁸DFLKSVTTLAMK³²⁹ | 2 | 677.38 | 1,352.74 |
| | 3 | 451.92 | 1,352.74 |
| ³⁵⁴TGYIQAPHK³⁶² | 2 | 513.79 | 1,025.57 |
| | 3 | 342.86 | 1,025.57 |
| ³⁵⁴IGYIQAPHKTLPVVFD³⁶⁹ | 2 | 899.50 | 1,796.98 |
| | 3 | 600.00 | 1,796.98 |
| ³⁵⁴IGYIQAPHKTLPVVFDSPRNRGLK³⁷⁷ | 5 | 542.31 | 2706.50^{\|} |
| ³⁶³TLPVVFDSPRNRGLK³⁷⁷ | 3 | 567.32 | 1,698.94*** |
| ³⁶³TLPVVFDSPRNRGLK³⁷⁷ | 3 | 610.34 | 1,827.98 |
| ³⁷⁹FPIKRVMGPD³⁸⁸ | 3 | 387.21 | 1,158.62 |
| ³⁸³RVMGPDFGYVTRGPQTGGISGLDSFGNLE⁴¹¹ | 3 | 1,009.83 | 3,026.45 |
| ³⁸³RVMGPDFGYVTRGPQTGGISGLDSFGNLE⁴¹¹ | 3 | 1,015.16 | 3,042.45^{†} |
| ³⁸⁹FGYVTRGPQTGGISGLDSFGNLE⁴¹¹ | 3 | 791.40 | 2,371.16 |
| ⁴⁴⁰SRQMHQALQDFLSAQQVQAPVK⁴⁶¹ | 4 | 628.33 | 2,509.29 |
| ⁴⁵⁰FLSAQQVQAPVK⁴⁶¹ | 2 | 658.37 | 1,314.73 |
| ⁴⁶²LYSDWLSVGHVDE⁴⁷⁴ | 2 | 760.36 | 1,518.70 |
| | 3 | 507.24 | 1,518.70 |
| ⁴⁷⁵FLSFVPAPDRK⁴⁸⁵ | 2 | 638.86 | 1,275.70 |
| | 3 | 426.24 | 1,275.70 |
| ⁴⁸⁶GFRLLLASPRSCYK⁴⁹⁹ | 3 | 556.64 | 1,666.90* |
| | 4 | 417.73 | 1,666.90* |
| ⁵⁰⁸GHGEALLFE⁵¹⁶ | 2 | 486.74 | 971.47 |
| ⁵¹²ALLFEGIK⁵¹⁹ | 2 | 445.77 | 889.53 |
| ⁵²⁶IKNILSNK⁵³³ | 3 | 310.53 | 928.57 |
| ⁵²⁶TKNILSNKTLRE⁵³⁷ | 2 | 714.93 | 1,427.84 |
| | 3 | 476.96 | 1,427.84 |
| | 4 | 357.97 | 1,427.84 |
| ⁵²⁸NILSNKTLRE⁵³⁷ | 3 | 396.56 | 1,186.67 |
| | 3 | 396.89 | 1,187.65 |
| | 2 | 594.34 | 1,186.67 |
| ⁵⁶²SDIIDIPQLFK⁵⁷² | 2 | 644.86 | 1,287.71 |
| ⁵⁸¹AFFPNMVNMLVLGK⁵⁹⁴ | 2 | 790.92 | 1,579.82 |
| | 3 | 527.62 | 1,579.82 |
| ⁵⁸¹AFFPNMVNMLVLGKHLGIPKPFGPVINGRCCLE⁶¹³ | 5 | 745.99 | 3,724.91* |
| | 5 | 746.59 | 3,727.91** |
| ⁵⁹⁵HILGIPKPFGPVINGRCCLE⁶¹³ | 3 | 722.04 | 2,163.11* |
| | 3 | 722.37 | 2,164.09** |
| ⁶¹⁵KVCSLLEPLGLQCTFIND⁶³² | 2 | 1,054.03 | 2,106.05* |
| | 3 | 703.02 | 2,106.05* |
| ⁶³³FFTYHIRHGE⁶⁴² | 2 | 653.82 | 1,305.63 |
| | 3 | 436.22 | 1,305.63 |
| | 4 | 327.41 | 1,305.62 |

*Modifications included: carbamidomethylation
**Modifications included: carbamidomethylation, deamidation
***Modifications included: citrullination
^{†}Modifications included: oxidation
^{|}Modifications included: deamidation

TABLE 3
Ions Detected from LC-MS/MS Analysis of R374Cit PAD4 digested with Lys-C and
Glu-C.

| | | Observed | Actual |
|---|---|---|---|
| Peptide sequence | Charge | Mass | Mass |
| ⁶¹KSTGSSTWPLDPGVE⁷⁵ | 2 | 780.8801 | 1,559.75 |
| | 3 | 520.9243 | 1,559.75 |
| ⁶²STGSSTWPLDPGVE⁷⁵ | 2 | 716.8336 | 1,431.65 |
| ⁹²VQISYYGPKTPPVK¹⁰⁵ | 3 | 526.2959 | 1,575.87 |
| ⁹²VQISYYGPKTPPVKALLYLTAVE¹¹⁴ | 3 | 850.8123 | 2,549.41 |
| ¹⁰⁶ALLYLTAVE¹¹⁴ | 2 | 496.7865 | 991.5584 |
| ¹¹⁵ISLCADITRTGK¹²⁶ | 2 | 667.858 | 1,333.70* |
| | 3 | 445.5746 | 1,333.70 |
| ¹⁸⁰MSLMTLSTKTPK¹⁹¹ | 3 | 446.5774 | 1,336.71 |
| ¹⁹²DFFTNHTLVLHVARSE²⁰⁷ | 4 | 472.2447 | 1,884.95 |
| ²²⁵CSVVLGPKWPSHYLMVPGGK²⁴⁴ | 4 | 553.7913 | 2,211.14* |
| ²⁴⁵HNMDFYVE²⁵² | 2 | 527.7189 | 1,053.42 |
| ²⁵³ALAFPDTDFPGLITLTISLLD²⁷³ | 2 | 1,117.10 | 2,232.19 |
| | 3 | 745.0708 | 2,232.19 |
| ²⁸²AVVFQDSVVFRVAPWIMTPNTQPPQE³⁰⁷ | 3 | 986.1743 | 2,955.50 |
| | 4 | 739.8812 | 2,955.50 |
| ²⁸⁸SVVFRVAPWIMTPNTQPPQE³⁰⁷ | 2 | 1,149.09 | 2,296.16 |
| | 3 | 766.3973 | 2,296.16 |
| | 4 | 575.0493 | 2,296.16 |
| ²⁸⁸SVVFRVAPWIMTPNTQPPQEVYACSIFE³¹⁵ | 3 | 1,089.87 | 3,266.58** |
| ³⁰⁸VYACSIFE³¹⁵ | 2 | 494.7257 | 987.4369* |
| ³¹⁸DFLKSVTTLAMK³²⁹ | 2 | 677.3763 | 1,352.74 |
| | 3 | 451.9195 | 1,352.74 |
| ³⁵⁴IGYIQAPHK362 | 3 | 342.8627 | 1,025.57 |
| ³⁵⁴IGYIQAPHKTLPVVFD³⁶⁹ | 2 | 899.4986 | 1,796.98 |
| | 3 | 600.001 | 1,796.98 |
| ³⁶³TLPVVFD³⁶⁹ | 2 | 395.7211 | 789.4276 |
| ³⁶³TLPVVFDSPRNRGLK³⁷⁷ | 3 | 567.3207 | 1,698.94*** |
| ³⁷⁰SPRNRGLKEFPIK³⁸² | 4 | 386.4741 | 1,541.87*** |
| ³⁸³RVMGPDFGYVTRGPQTGGISGLDSFGNLE⁴¹¹ | 3 | 1,009.83 | 3,026.46 |
| | 4 | 757.6212 | 3,026.46 |
| ³⁸⁹FGYVTRGPQTGGISGLDSFGNLE⁴¹¹ | 2 | 1,186.58 | 2,371.15 |
| | 3 | 791.3882 | 2,371.15 |
| ⁴⁴⁰SRQMHQALQDFLSAQQVQAPVK⁴⁶¹ | 4 | 628.3287 | 2,509.29 |
| ⁴⁵⁰FLSAQQVQAPVK⁴⁶¹ | 2 | 658.371 | 1,314.73 |
| ⁴⁶²LYSDWLSVGHVDE⁴⁷⁴ | 2 | 760.3563 | 1,518.70 |
| | 3 | 507.2402 | 1,518.70 |
| ⁴⁷⁵FLSFVPAPDRK⁴⁸⁵ | 2 | 638.856 | 1,275.70 |
| | 3 | 426.2394 | 1,275.70 |
| ⁴⁸⁶GFRLLLASPRSCYK⁴⁹⁹ | 3 | 556.639 | 1,666.90* |
| | 4 | 417.7319 | 1,666.90* |
| ⁵⁰⁸GHGEALLFE⁵¹⁶ | 2 | 486.743 | 971.4714 |
| ⁵¹²ALLFEGIK⁵¹⁹ | 2 | 445.7715 | 889.5285 |
| ⁵²⁶IKNILSNKTLRE⁵³⁷ | 2 | 7 14.9295 | 1,427.84 |
| | 3 | 476.9563 | 1,427.84 |
| | 4 | 357.9686 | 1,427.84 |
| ⁵²⁸NILSNKTLRE⁵³⁷ | 2 | 594.3407 | 1,186.67 |
| | 3 | 396.5632 | 1,186.67 |
| ⁵³⁸HINSFVERCIDWNRE⁵⁵¹ | 3 | 621.2845 | 1,860.83* |
| | 4 | 466.2158 | 1,860.83* |
| ⁵⁶²SDIIDIPQLFK⁵⁷² | 2 | 644.8606 | 1,287.71 |
| | 3 | 430.2436 | 1,287.71 |
| ⁵⁷⁹AEAFFPNMVNMLVLGK⁵⁹⁴ | 2 | 890.9604 | 1,779.91 |
| | 3 | 594.3103 | 1,779.91 |
| ⁵⁸¹AFFPNMVNMLVLGK⁵⁹⁴ | 2 | 790.9189 | 1,579.82 |
| | 3 | 527.6149 | 1,579.82 |
| ⁵⁸¹AFFPNMVNMLVLGKHLGIPKPFGPVINGRCCLE⁶¹³ | 5 | 745.99 | 3,724.91* |
| ⁵⁹⁵HILGIPKPFGPVINGRCCLE⁶¹³ | 3 | 722.0433 | 2,163.11* |
| ⁶¹⁵KVCSLLEPLGLQCTFIND⁶³² | 2 | 1,054.03 | 2,106.05* |
| | 3 | 703.023 | 2,106.05* |
| ⁶¹⁶VCSLLEPLGLQCTFIND⁶³² | 3 | 660.326 | 1,977.96* |
| ⁶¹⁶VCSLLEPLGLQCTFINDFFTYHIRHGE⁶⁴² | 4 | 817.8986 | 3,267.57** |
| ⁶³³FFTYHIRHGE⁶⁴² | 2 | 653.8193 | 1,305.62 |
| | 3 | 436.2157 | 1,305.62 |
| | 4 | 327.4137 | 1,305.62 |

*Modifications included: carbamidomethylation
**Modifications included: carbamidomethylation, deamidation
***Modifications included: citrullination

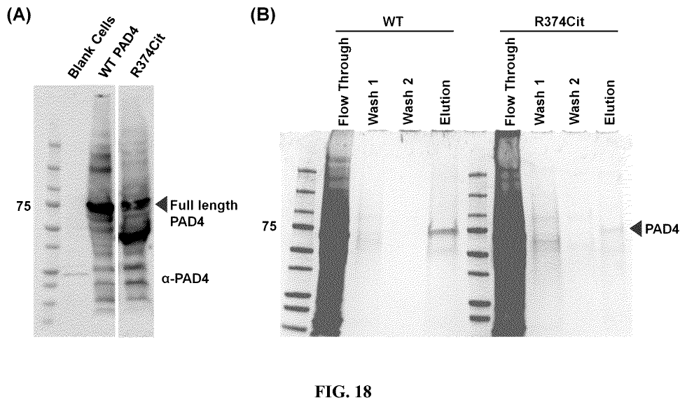

Notably, it was found that PAD4 expression does not cause any cytotoxicity in the HEK293T cells (FIG. 14B). Using a similar procedure, wild-type PAD4 and the R374Cit mutant were expressed and purified from EXPI293F cells that grow in suspension and are highly scalable expression system (FIG. 18).

### Activity of WT, R372Cit, R374Cit and Autocitrullinated PAD4

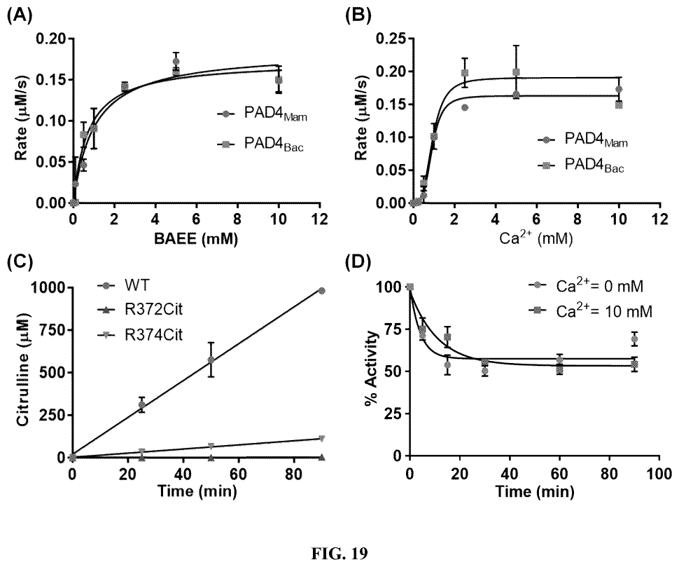

The biochemical activity and calcium dependence of WT PAD4 expressed from HEK293T cells (PAD4_{Mam}) and E. coli (PAD4_{Bac}) was first ensured to be similar (FIG. 19a, FIG. 19b and Table 4). Next determined were kinetic values for the R372Cit and R374Cit mutants. Notably, both WT and the R374Cit mutant exhibited a time-dependent increase in citrulline production (FIG. 19c). By contrast, the R372Cit mutant produced only a negligible amount of citrulline after 90 min. Furthermore, the rate of citrulline production, indicated by the slope of the straight line, is significantly lower for the R374Cit mutant than WT PAD4 (FIG. 19c). In agreement, the k_{cat}/Kₘ values of the R374Cit and R372Cit mutants are 9- and 181-fold lower than that for WT PAD4 (FIG. 3d and Table 4).

TABLE 4
Steady-state kinetic parameters for wild-type
PAD4_{Bac}, wild-type PAD4_{Mam}, R372Cit and R374Cit mutants.

| | k_{cat} | Kₘ | k_{cat}/Kₘ | K_{0.5} |
|---|---|---|---|---|
| Enzyme | (S⁻¹) | (mM) | (M⁻¹S⁻¹) | (mM) |
| PAD4_{Bac} | 3.4 ± 0.2 | 0.7 ± 0.1 | 4740 ± 260 | 0.9 ± 0.10 |
| PAD4_{Mam} | 3.7 ± 0.1 | 1.1 ± 0.2 | 3620 ± 810 | 0.9 ± 0.05 |
| R372Cit | — | — | 20 ± 2 | — |
| R374Cit | 0.33 ± 0.01 | 0.84 ± 0.06 | 390 ± 40 | — |

k_{cat}: turnover number; Kₘ: Michaelis-Menten constant; k_{cat}/Kₘ: catalytic efficiency; K_{0.5}: Ca²⁺ concentration for half-maximal activity

These in vitro results led us to investigate the activity of WT and R374Cit PAD4 in live cells. Specifically, the two enzymes in HEK293T cells were overexpressed and evaluated for their ability to citrullinate histone H3. Treatment of PAD4-overexpressing HEK293T cells with calcium and a calcium ionophore, i.e. ionomycin, followed by western blot analysis indicated that WT PAD4 is 6-times more active than the R374Cit mutant for the citrullination of histone H3, consistent with the in vitro results (FIG. 3e).

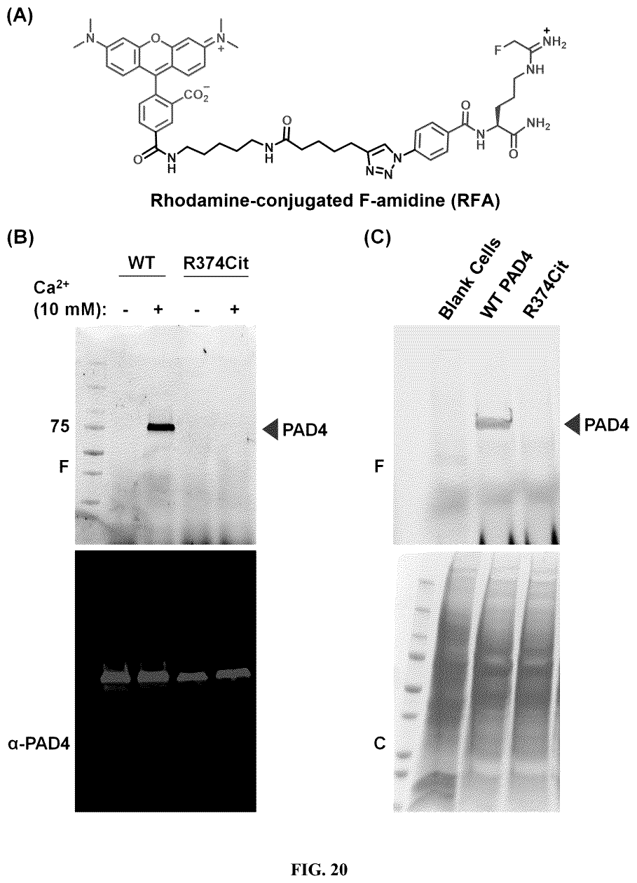

In regard to why the activity of these Cit-containing mutants is lower than WT PAD4, kinetic studies indicated that R374Cit mutant possesses a similar Kₘ, but a 10-fold lower k_{cat} than WT PAD4 (Table 4), suggesting a slow conversion of substrate to product. Furthermore, RFA, a PAD-targeted activity-based probe that covalently modifies the active site cysteine, C645, fluorescently labeled only WT PAD4 when tested with both purified enzymes and enzyme-containing cell lysates (FIG. 20). (Luo, et al. 2006 J. Am. Chem. Soc. 128, 14468-14469.)

To investigate this possibility that citrullination may induce local conformational changes within the active site, leading to very slow or no reaction between C645 and the guanidium group of substrate, BAEE or the fluoroacetamidine warhead on RFA, a thermal shift assay was performed in the presence of a PAD4-selective ligand, GSK199, which binds to an allosteric pocket near the active site and H-bonds to both D473 and H471. (Lewis, et al. 2015 Nat. Chem. Biol. 11, 189-191.)

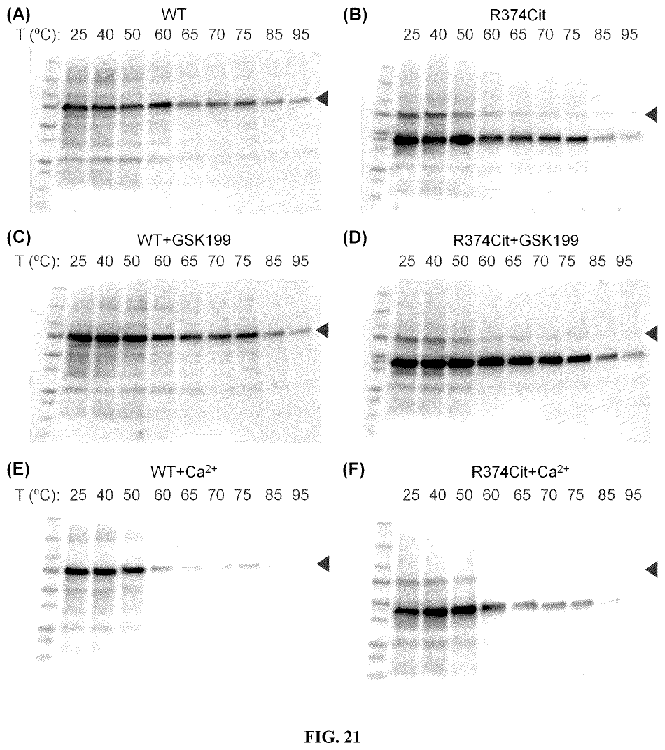

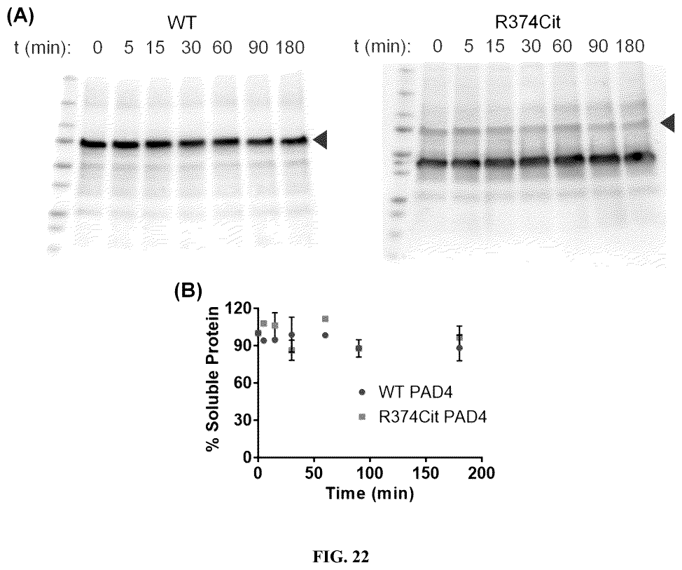

Using this assay, the melting temperatures (Tₘ) of WT and R374Cit PAD4 were found to be 62.9 and 54.6° C., respectively (FIG. 3f and FIG. 21). Despite having a lower Tₘ, the R374Cit mutant is as stable as WT PAD4 at 37° C. (FIG. 22). As expected, GSK199 increased the Tₘ of WT PAD4, however, it did not increase the Tₘ of the R374Cit mutant (FIG. 3f). These results indicate that GSK199 binds poorly to the R374Cit mutant, likely due to local conformational changes around the active site. However, the overall folding of the mutant is the same as WT since both the WT and R374Cit proteins exhibit a similar ΔTₘ in the presence of calcium (FIG. 3f).

### Quantitative Proteomics of PAD4 Autocitrullination

As discussed earlier, conflicting reports indicated that autocitrullination can either inactivate the enzyme or have no effect. Since the present results suggest that autocitrullination should decrease PAD4 activity, autocitrullinated PAD4 was regenerated by incubating the enzyme in the presence of 10 mM CaCl₂. Consistent with previous observations, autocitrullinated PAD4 exhibits similar activity to control PAD4 (incubated in the absence of CaCl₂) (FIG. 19d). The activity loss for both autocitrullinated and control PAD4 in this experiment is likely due to the oxidation of C645 over time. Given that autocitrullination does not decrease enzyme activity, but citrullination of R372 and R374 does, two questions need to be answered. Are R372 and R374 the preferred sites of autocitrullination? Also, what fraction of PAD4 gets autocitrullinated? If only a small fraction of PAD4 is autocitrullinated, then this process should not impact the activity of uncitrullinated enzyme.

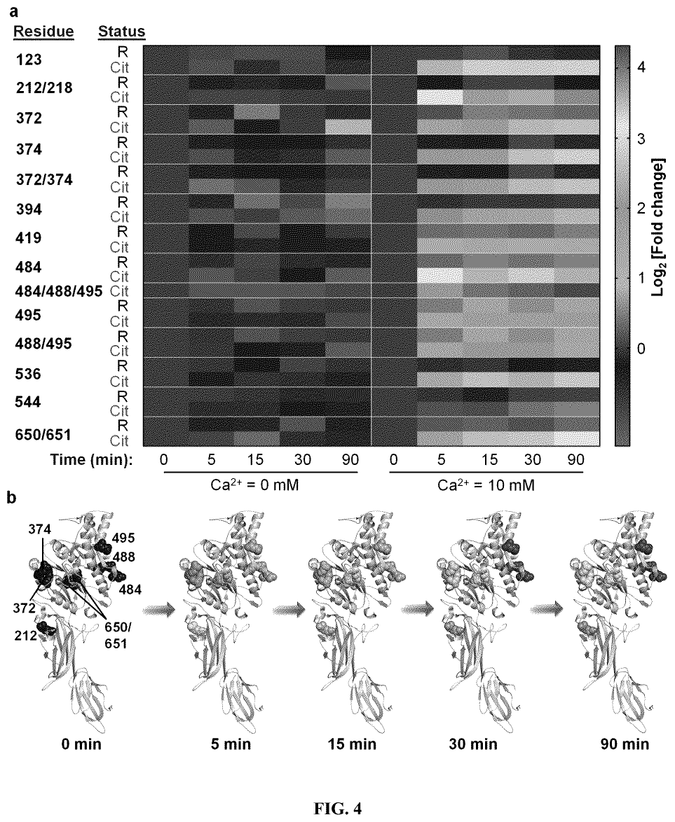

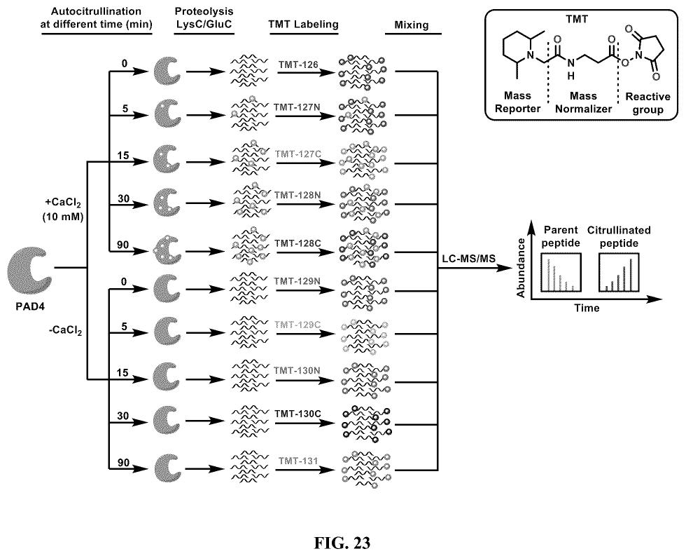

To answer these questions, a quantitative proteomics approach was taken. PAD4 was autocitrullinated for various times, and digested with Glu-C and Lys-C to maximize peptide coverage. The resulting peptides were then labeled with tandem mass tags (TMT) and were subjected to tandem mass analysis (FIG. 23). Enzyme incubated in the absence of calcium was used as the negative control. From this analysis, 13 unique citrullination sites on PAD4 were identified (FIG. 4a, Table 1 and Table 5). Notably, these exclude the previously reported R156, R205, R383, R609 and R639 and include two new sites, R650 and R651. Although both R372 and R374 residues show a time-dependent increase in citrullination, citrullination of arginines 212/218, 484/488/495 and 650/651 occurs at much higher rate. For example, the extent of citrullination at arginines 212/218 and 484/488/495 is significantly higher at 5 min than that at the 372 and 374 sites after 90 min (FIG. 4a and FIG. 4b). These observations indicate that arginines 372 and 374 are not the preferred sites of autocitrullination. Additionally, none of the observed autocitrullination sites exhibited a marked decrease in the arginine-containing parent peptide levels, which indicates that only a minor fraction of PAD4 undergoes autocitrullination, further explaining its nominal impact on the enzyme activity. Nonetheless, these results showcase how this technology provides the ability to systematically characterize the behavior of individual citrullinated isoforms of any protein—both in vitro and in living cells—providing a powerful new approach to understand the biology of this PTM.

TABLE 5
Normalized^{†} fold changes of the peptides containing Arg and Cit at various
autocitrullination sites with increasing time in the absence and presence of calcium.

| | | CaCl₂ (0 mM) | | | | | CaCl₂ (10 mM) | | | | |
|---|---|---|---|---|---|---|---|---|---|---|---|
| | | 0 | 5 | 15 | 30 | 90 | 0 | 5 | 15 | 30 | 90 |
| Site | Status | min | min | min | min | min | min | min | min | min | min |
| 123 | Arg | 1 | 1.060198 | 1.099362 | 1.086233 | 0.847332 | 1 | 1.015601 | 1.108801 | 0.955283 | 0.90346 |
| | Cit | 1 | 1.111622 | 0.681444 | 1.061178 | 0.931525 | 1 | 5.35171 | 7.061624 | 6.900336 | 7.226682 |
| 212/218 | Arg | 1 | 0.763923 | 0.717972 | 0.867539 | 0.447513 | 1 | 0.844928 | 1.023137 | 1.030968 | 0.845279 |
| | Cit | 1 | 1.043454 | 0.542113 | 0.587774 | 0.614152 | 1 | 8.693879 | 13.17746 | 12.295 | 14.45336 |
| 372 | Arg | 1 | 0.883519 | 1.368884 | 0.993322 | 0.918658 | 1 | 1.112136 | 1.391525 | 1.316463 | 1.254982 |
| | Cit | 1 | 1.176907 | 0.858565 | 0.584389 | 1.82134 | 1 | 3.093736 | 4.228072 | 5.637283 | 6.04189 |
| 374 | Arg | 1 | 0.875998 | 0.849096 | 0.846745 | 0.939523 | 1 | 0.869947 | 0.809442 | 1.046085 | 0.90485 |
| | Cit | 1 | 1.095559 | 0.741919 | 0.930278 | 1.176091 | 1 | 4.169863 | 4.267329 | 6.175974 | 7.094331 |
| 372/374 | Arg | 1 | 0.875998 | 0.849096 | 0.846745 | 0.939523 | 1 | 0.869947 | 0.809442 | 1.046085 | 0.90485 |
| | Cit | 1 | 1.294953 | 1.149787 | 0.90417 | 1.006956 | 1 | 4.018527 | 4.30695 | 5.81589 | 6.233317 |
| 394 | Arg | 1 | 0.901459 | 1.284909 | 1.065847 | 1.397647 | 1 | 0.942636 | 0.987145 | 0.965936 | 0.99424 |
| | Cit | 1 | 1.107009 | 1.01607 | 1.156688 | 1.256723 | 1 | 1.528377 | 1.672493 | 1.699763 | 1.930088 |
| 419 | Arg | 1 | 0.827406 | 1.071773 | 0.860154 | 0.609628 | 1 | 1.271619 | 1.311302 | 1.23086 | 1.387031 |
| | Cit | 1 | 0.832199 | 0.72951 | 0.803851 | 0.869545 | 1 | 2.361985 | 2.763826 | 2.367449 | 2.578741 |
| 484 | Arg | 1 | 0.92959 | 1.047052 | 0.926588 | 0.899794 | 1 | 1.242001 | 1.414214 | 1.310393 | 1.420436 |
| | Cit | 1 | 1.138131 | 0.573024 | 0.833161 | 1.173919 | 1 | 9.210844 | 11.60494 | 10.33882 | 11.87619 |
| 484/488/495 | Cit | 1 | 1.123889 | 0.467056 | 0.470848 | 0.52912 | 1 | 13.48546 | 16.22335 | 17.79418 | 19.97329 |
| 495 | Arg | 1 | 0.95418 | 1.118062 | 1.007654 | 0.982457 | 1 | 1.381913 | 1.689582 | 1.505247 | 1.691262 |
| | Cit | 1 | 0.903753 | 0.697533 | 0.91088 | 1.074749 | 1 | 2.757447 | 3.363586 | 3.294364 | 3.547166 |
| 488/495 | Arg | 1 | 0.95418 | 1.118062 | 1.007654 | 0.982457 | 1 | 1.381913 | 1.689582 | 1.505247 | 1.691262 |
| | Cit | 1 | 0.972655 | 0.82932 | 0.768438 | 1.170128 | 1 | 2.909961 | 4.05865 | 4.153517 | 4.771138 |
| 536 | Arg | 1 | 0.965267 | 0.835088 | 1.035026 | 0.652176 | 1 | 0.932817 | 0.970859 | 0.828171 | 0.870752 |
| | Cit | 1 | 0.808321 | 0.960595 | 0.93045 | 0.872363 | 1 | 4.808432 | 6.058245 | 5.274009 | 6.644835 |
| 544 | Arg | 1 | 0.997692 | 0.963707 | 0.905216 | 0.692555 | 1 | 0.960062 | 0.872081 | 1.013397 | 1.022755 |
| | Cit | 1 | 0.924236 | 0.929161 | 0.844401 | 0.875796 | 1 | 1.113087 | 1.076738 | 1.301342 | 1.410298 |
| 650/651 | Arg | 1 | 0.872161 | 0.893992 | 1.115739 | 0.769148 | 1 | 1.148698 | 1.202191 | 1.109467 | 1.248331 |
| | Cit | 1 | 1.038139 | 0.379893 | 0.671116 | 0.728681 | 1 | 4.505436 | 6.083915 | 6.520579 | 8.805063 |

^{†}Ratios for the calcium- treated and untreated data set were normalized against the respective 0 min time-point.

### EXAMPLES

### Materials and General Method

N^{α}-Fmoc-N^{δ}-L-Ornithine hydrochloride, HBTU, HOBt and other Fmoc-protected amino acids were purchased from Chem-Impex International Inc. 1-(isocyanatomenthyl)-2-nitrobenzene (1 M solution in toluene) was purchased from Ellanova Laboratories. Triethylamine, trifluoroacetic acid, anhydrous dichloromethane, anhydrous dimethylformamide, piperidine and HPLC-grade acetonitrile were bought from Sigma-Aldrich. Halt protease inhibitor cocktail (EDTA-free), Universal nuclease, Ni-NTA resin, Pierce™ Peptide Desalting Spin Columns (Catalogue No 89852), Pierce™ Quantitative Fluorometric Peptide Assay kit (Catalogue No 23290) and TMT10plex™ Isobaric Label Reagent (Catalogue No 90110) were obtained from ThermoFisher Scientific. Deuterated solvents were purchased from Cambridge Isotope Laboratories. Plasmid purification kit was bought from Bio Basic Canada Inc. The TOP10 E. coli strain was used for plasmid construction and propagation. The cells were grown in LB liquid medium with 100 μg/ml ampicillin or 50 mg/ml kanamycin. Primers were synthesized by Integrated DNA Technologies (Coralville, IA). Restriction enzymes (NEB, Beverly, MA), Phusion Hot Start II DNA polymerase (Fisher Scientific, MA) and T4 DNA ligase (Enzymatics, Beverly, MA) were used for plasmid construction following manufacturers' protocols. White light and fluorescence imaging of HEK293T cells expressing the EGFP-39-TAG reporter were performed using a Zeiss AX10 microscope. Rabbit polyclonal anti-PAD4 (catalogue no. ab50332) was obtained from Abcam. EXPI293F cells, EXPI293™ expression medium and ExpiFectamine™ 293 Transfection Kit were obtained from Gibco. Dulbecco's Modified Eagle's medium (DMEM), fetal bovine serum (FBS) and Antibiotic-Antimycotic (100×) solution were obtained from Gibco and were used for HEK293T cell maintenance. Mass spec grade Lys-C (Catalogue No VA117A) and sequencing grade Glu-C (Catalogue No V165A) was obtained from Promega. ¹H and ¹³C NMR spectra were recorded in d₆-DMSO as solvent using a Bruker 500 MHz NMR spectrometer. Chemical shift values are cited with respect to SiMe₄ (TMS) as the internal standard. All the compounds were purified by reverse-phase HPLC using a semi-preparative C₁₈ column (Agilent, 21.2×250 mm, 10 μm) and a water/acetonitrile gradient supplemented with 0.05% trifluoroacetic acid. Fluorographs were recorded using a Typhoon scanner with excitation/emission maxima of ˜546/579, respectively. Wild-type PAD4 obtained from a bacterial expression system (PAD4_{Bac}) was expressed and purified as reported earlier. (Slack, et al. Biochemistry 50, 3997-4010 (2011).)

### Synthesis of SM60

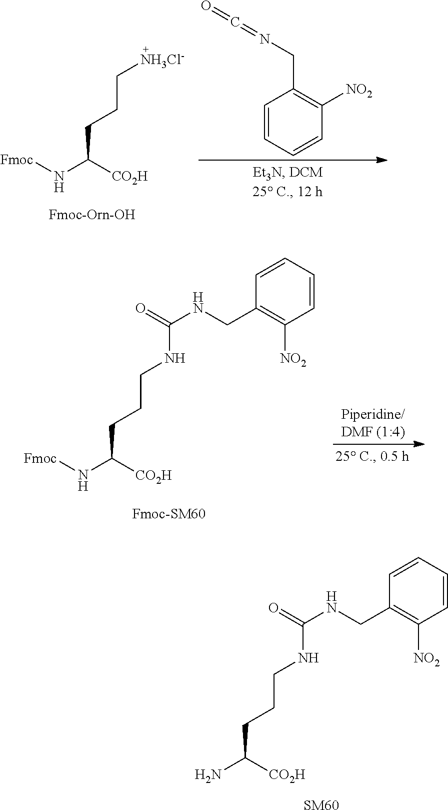

Fmoc-Orn-OH (1 g, 2.6 mmol) was suspended in anhydrous dichloromethane and triethylamine (0.7 mL, 5.2 mmol) was added to it. 1-(isocyanatomenthyl)-2-nitrobenzene (1 M solution in toluene) (2.6 mL, 2.6 mmol) was then added dropwise and the reaction mixture was stirred at room temperature for 12 h. Excess triethylamine and dichloromethane was then evaporated under reduced pressure to afford a yellowish brown semisolid that was used for the subsequent step without further purification. The crude product was dissolved in 1:4 piperidine/dimethylformamide (10 mL) and was stirred at room temperature for 30 min. The reaction mixture was then vigorously stirred with excess hexane and the hexane layer was decanted off. Washing with hexane was repeated several times to remove most of the dimethylformamide. The pale yellow semisolid obtained thereafter was purified by reverse phase HPLC using a pre-packed C18 column and a water/acetonitrile (supplemented with 0.05% trifluoroacetic acid) gradient as eluent to afford SM60 as a white solid (overall yield: 60%). SM60 was thoroughly characterized with ¹H and ¹³C NMR spectroscopy and Mass spectrometry. ¹H NMR (DMSO-d₆) δ (ppm): 8.18 (s, 3H), 7.94 (dd, J=9 Hz, 1H), 7.64-7.67 (m, 1H), 7.43-7.48 (m, 2H), 6.48 (t, J=5 Hz, 1H), 6.24 (t, J=5 Hz, 1H), 4.4 (d, J=10 Hz, 2H), 3.85 (s, 1H), 2.93-2.97 (m, 2H), 1.61-1.75 (m, 2H), 1.40-1.49 (m, 1H), 1.31-1.39 (m, 1H); ¹³C NMR (DMSO-d₆) δ (ppm): 171.5, 158.5, 148.3, 136.6, 134.2, 130.0, 128.4, 124.9, 52.3, 40.7, 39.1, 28.0, 26.2; ESI-MS (m/z) calculated for C₁₃H₁₈N₄O₅ [M+H]⁺: 311.14, found 311.20.

### Synthesis of SM70

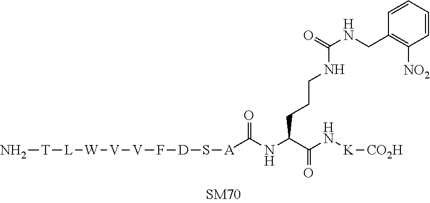

SM70 was synthesized using an automated solid-phase peptide synthesizer (PS3, Protein Technologies, Inc.) by following the manufacturer's protocol. Briefly, Fmoc-Lys(Boc)-Wang resin (350 mg, 0.2 mmol) was taken in a 30 mL glass reaction vessel, and Fmoc-SM60 (425 mg, 0.8 mmol), Fmoc-Ala-OH (249 mg, 0.8 mmol), Fmoc-Ser(tBu)-OH (306 mg, 0.8 mmol), Fmoc-Asp(OtBu)-OH (329 mg, 0.8 mmol), Fmoc-Phe-OH (310 mg, 0.8 mmol), Fmoc-Val-OH (272 mg, 0.8 mmol), Fmoc-Val-OH (272 mg, 0.8 mmol), Fmoc-Trp(Boc)-OH (421 mg, 0.8 mmol), Fmoc-Leu-OH (283 mg, 0.8 mmol) and Fmoc-Thr-OH (318 mg, 0.8 mmol) were taken in separate amino acid vials. HBTU (303 mg, 0.8 mmol) and HOBt (108 mg, 0.8 mmol) were then added to each amino acid vial. N-methylmorpholine (0.4 M in DMF) was used as a base for the activation of the carboxylic acid group with HBTU and HOBt. The N-terminal Fmoc protecting group on each amino acid was removed with 20% piperidine in DMF. Each peptide coupling reaction was carried out for 1 h. Once all the amino acids were coupled, the resin was transferred to a synthetic column, thoroughly washed with DMF and DCM, and the peptide was cleaved from the resin by treating with a cleavage cocktail (95% trifluoroacetic acid, 4.5% triisopropylsilane, 0.5% water, 10 mL) at room temperature for 30 min with constant mixing. The flow-through was collected and the resin was washed 2-3 times with trifluoroacetic acid. The combined flow through and trifluoroacetic acid washes were then treated with 8-10 times excess cold diethyl ether to precipitate the peptide. Excess ether and trifluoroacetic acid was slowly evaporated by purging nitrogen. The crude peptide was purified by reverse phase HPLC using a pre-packed C18 column and a water/acetonitrile (supplemented with 0.05% trifluoroacetic acid) gradient to afford SM70 as a white solid (overall yield: 15%). SM70 was characterized by ESI mass spectrometry, ESI-MS (m/z) calculated for C₆₉H₁₀₀N₁₆o₁₉ [M]⁺: 1456.74, found 1456.60.

### Construction of Plasmids

The previously reported pAcBac3-EcLeuTAG-EGFP-39-TAG plasmid was used to construct additional plasmids. (Zheng, et al. Biochemistry 57, 441-445 (2018).) EGFP-39-TAG was replaced with WT PAD4 using SfiI restriction site to create pAcBac3-EcLeuPLRS1TAG-PAD4WT. For incorporation of SM60, TAG nonsense codon was introduced at the desired sites by site-directed mutagenesis based on the pAcBac3-EcLeuPLRS1TAG-PAD4WT plasmid. pIDTSMART eRF1 E55D was generated following literature. (Schmied, et al. J. Am. Chem. Soc. 136, 15577-15583 (2014).)

### List of Primers

PAD4WT-Forward:
ATTATTAGAATTGGCCAAGGAGGCCACCATGGACTACAAGGACGACGAC
GACAAG
PAD4WT-Reverse:
ATTATTAGAATTCGGCCTTAGAGGCCTCAGTGGTGGTGGTGGTGGTGGT
GGTGGTGGTGGTGGTGGGGCACCATGTTCCACCACTTGAA
PAD4 R372TAG Inner Forward:
GTCTTCGACTCTCCTTAGAACTAGGGCCTGAAG
PAD4 R372TAG Inner Reverse:
CTTCAGGCCCTAGTTCTAAGGAGAGTCGAAGAC
PAD4 R374TAG Inner Forward:
GTCTTCGACTCTCCTAGGAACTAGGGCCTGAAG
PAD4 R374TAG Inner Reverse:
CTTCAGGCCCTAGTTCCTAGGAGAGTCGAAGAC

### HEK293T Cell Culture and Transfection

HEK293T cells were maintained at 37° C. in a humidified incubator supplemented with 5% CO₂. Cells were seeded at 9×10⁶ cells per 10 cm plate 24 h before transfection. EGFP and WT PAD4 transfections were performed by incubating 10 μg plasmid DNA, 50 μL PEI (1 mg/mL; Polysciences, Warrington, PA), and 180 μL DMEM for 10 min at room temperature, followed by adding the solution dropwise to the culture medium of the cells. For SM60 incorporation into PAD4 at positions 372 and 374, 12 μg of PAD4 R372TAG or PAD4 R374TAG and 8μg of pIDTSMART eRF1 E55D plasmids were incubated with a mixture of 100 μL PEI and 180 μL DMEM for 10 min at room temperature before adding to cells. SM60 was added at the same time to a final concentration of 1 mM, and 2 mM sodium butyrate was added to enhance protein expression.

### EGFP Fluorescence Analysis

EGFP fluorescence was analyzed 48 h after transfection. DMEM was exchanged with PBS and the plates were irradiated at 365 nm (120 Watt, 10 cm×10 cm LED array; Larson Electronics) for 75 s at 4° C. to decage SM60. Cells were then harvested and resuspended in 600 μL CelLytic M buffer (Sigma, St. Louis, MO) with Halt Protease inhibitor Cocktail (Thermo Scientific, Waltham, MA) and Pierce Universal Nuclease for Cell Lysis (Fisher Scientific, Hampton, NH). Lysate were clarified by centrifugation at 16,000×g for 10 min and 100 μL of supernatant was transferred to a clear-bottom 96-well plate for fluorescence measurement following previously described protocol. All the experiments were performed at least in duplicate.

### Cytotoxicity of SM60 in HEK293T Cells

HEK293T cells were seeded (2×10⁴ cells/well) on a 96-well plate and grown in DMEM (supplemented with 10% heat-inactivated fetal bovine serum, 100 units/mL penicillin, 100 μg/mL streptomycin) for 48 h. Cells were then treated with DMSO (for control) or various concentrations of SM60. In a separate experiment, cells were briefly (3 min) irradiated with 365 nm UV following SM60 treatment and were allowed to grow at 37° C. for 48 h. Cell viability was measured using CellTiter-Blue® (Promega) by following the manufacturer's protocol. Equation 1,

Y=Bottom+(Top−Bottom)/[1+10^{((log EC50-X)*Hillslope)}] (1),

was used to fit a ten-point dose-response curve to determine the EC₅₀ values for inhibition of cell-proliferation using GraphPad Prism 8.0. Top and Bottom are plateaus of the dose-response curve, X is the log of SM60-concentration, Hillslope is the slope factor or Hill slope. All the experiments were performed in triplicate.

### Overexpression of Wild-Type and Mutant (R374Cit) PAD4 in EXPI293F Cells

EXPI293F cells were cotransfected with the engineered LeuRS/tRNALeu/PAD4 and eRF genes using ExpiFectamine™ 293 transfection kit by following the manufacturer's protocol. Briefly, EXPI293F cells were grown to 2.9×10⁶ density in the EXPI293 expression medium (9 mL) at 37° C. under 5% CO₂ atmosphere. pAcBac3 Plasmid (6 μg) encoding the genetically engineered LeuRS, tRNA^{Leu} and WT PAD4 or mutant PAD4 (containing TAG mutation at 374 position) and the plasmid encoding release factor, pIDTSMART eRF1 E55D (4 μg), were resuspended in 0.5 mL opti-MEM™ I reduced serum media. ExpiFectamine™ (27 μL) was also resuspended in 0.5 mL opti-MEM™ media and then the plasmid mixture was slowly added to ExpiFectamine™ solution. This mixture was incubated at room temperature for 20 min to form the DNA polyplex. Then the DNA polyplex (1 mL) was slowly transferred to the EXPI293F cell suspension (9 mL) and SM60 (100 μL of 100 mM stock in DMSO, 1 mM final) was added to the medium. The cells were then incubated at 37° C. under 5% CO₂ atmosphere for 24 h with constant shaking. Transfection enhancers 1 (50 μL) and 2 (500 μL) (supplied with the ExpiFectamine™ 293 transfection kit) were then added to the cell suspension and the cells were further grown at 37° C. under 5% CO₂ atmosphere for 48 h with constant shaking. Cells were then harvested, washed with cold Dulbecco's Phosphate-Buffered Saline (DPBS), resuspended in 5 mL DPBS and were taken in a cell culture dish (100 mm×20 mm, Corning). Cells were then irradiated with 365 nm light for 5 minute using a photoreactor (Luzchem) containing 14 UV-A lamps (8 W each). After UV-A irradiation, cells were harvested, resuspended in 1 mL in DPBS (containing 1× Halt protease inhibitor cocktail) and lysed using a probe sonicator. Overexpression of wild-type and mutant PAD4 was confirmed by western blot analysis of the EXPI293F cell lysate using rabbit polyclonal anti-PAD4 antibody.

### Purification of Wild-Type and Mutant PAD4 from HEK293T Cells

Cells from a 10 cm plate were harvested 48 h after transfection. For cells that overexpressed proteins containing SM60, media was exchanged with PBS and the plates were irradiated at 365 nm (120 Watt, 10 cm×10 cm LED array (Larson Electronics)) for 75 s at 4° C. to decage SM60 right before harvesting. The cells were resuspended in 600 μL CelLytic M buffer (Sigma, St. Louis, MO) with Halt protease inhibitor cocktail (Thermo Scientific, Waltham, MA) and Pierce universal nuclease for cell lysis (Fisher Scientific, Hampton, NH). After a 10 min incubation at room temperature, 1.2 mL of equilibration buffer (20 mM Na₂HPO₄, 300 mM NaCl, 10 mM imidazole pH 7.4) was added. Lysate was clarified by centrifugation at 16,000×g for 10 min at 4° C. The clarified cell-free extract was subjected to Ni-NTA affinity chromatography using HisPur resin (Fisher Scientific, Hampton, NH) following the manufacturer's protocol.

### Purification of Wild-Type and Mutant PAD4 from EXPI293F Cells

Following UV-A irradiation, cells were resuspended in lysis buffer (20 mM TRIS-HCl pH 8.0, 400 mM NaCl, 10 mM imidazole, 2 mM DTT, 10% glycerol, 1× protease inhibitor cocktail, 1× universal nuclease) and lysed using a probe sonicator. The lysate was centrifuged at 18000×g for 20 min at 4° C. and the supernatant was incubated with Ni-NTA agarose beads (prewashed with the lysis buffer) for 30 min at 4° C. on an end-over-end shaker. The beads were then transferred to a synthetic column and washed sequentially with buffer 1 (20 mM TRIS-HCl pH 8.0, 400 mM NaCl, 50 mM imidazole, 2 mM DTT, 10% glycerol) and buffer 2 (20 mM TRIS-HCl, pH 8.0, 400 mM NaCl, 75 mM imidazole, 2 mM DTT, 10% glycerol). Finally, PAD4 was eluted from the beads using elution buffer (20 mM TRIS-HCl pH 8.0, 500 mM NaCl, 300 mM imidazole, 2 mM DTT, 10% glycerol). Eluted PAD4 was then dialyzed against 20 mM TRIS-HCl, pH 8.0, 500 mM NaCl, 2 mM DTT, 10% glycerol using a dialysis cassette with a molecular weight cut off 3.5 kDa.

### Digestion of the PAD4R372Cit and PAD4R374Cit Mutants, and LC-MS/MS Analysis

The R372Cit and R374Cit mutants (15 μg) were independently resuspended in 20 mM TRIS-HCl (200 μL, pH 7.4) and then trichloroacetic acid (TCA, 20% final) added to the samples. The resultant cloudy mixture was vortexed vigorously and was kept at −20° C. for 30 min to precipitate the protein. Then the mixture was centrifuged at 15,000 rpm for 30 min at 4° C. The pellet was washed with cold acetone, resuspended in 30 μL of 8 M urea in PBS (pH 7.4) with sonication and 70 μL of 100 mM ammonium bicarbonate was added to the solution. Then 1.5 μL of 1 M DTT was added and the solution was incubated at 65° C. for 20 min. 2.5 μL of freshly-prepared 500 mM iodoacetamide was added and the solution was incubated at room temperature for 30 min in dark. The alkylation reaction was diluted by the addition of 120 μL PBS (pH 7.4). Lys-C (1 μL of a 1 μg/μL solution, reconstituted in water) and Glu-C (2 μL of a 0.5 μg/μL solution, reconstituted in water) was then added to the samples and the mixture was incubated at 37° C. for 16 h. The proteolysis reaction was terminated by adding 10 μL of formic acid. The peptide mixture was then desalted using Pierce™ desalting C18 spin columns following the manufacturer's protocol and the samples were analysed by LC-MS/MS as described below.

### Cytotoxicity of PAD4 Expression in HEK293T Cells

HEK293T cells were seeded (2×10⁴ cells/well) on 96-well plate and were allowed to grow in DMEM (supplemented with 10% heat-inactivated fetal bovine serum, 100 units/mL penicillin, 100 μg/mL streptomycin) for 48 h. Cells were then transfected with appropriate plasmids (120 ng PAD4 plasmid and 80 ng pIDTSMART eRF1 E55D for each well) using Lipofectamine 2000 (500 ng for each well) in Opti-MEM medium (Gibco). After 5 h, the medium was changed to DMEM and DMSO (for WT PAD4) or SM60 (0.25, 5 and 1 mM for R374Cit PAD4) was added. Cells were then allowed to grow at 37° C. for 48 h. Cell viability was measured before and after photodecaging (by UV treatment for 3 min) using CellTiter-Blue® (Promega) by following the manufacturer's protocol. Untransfected cells served as the control. All the experiments were performed in triplicate.

### Time-Dependent Citrulline Production by Wild-Type, R372Cit and R374Cit PAD4 Purified from the Mammalian Expression System

PAD4 (6 μL of a 1 μM stock, 100 nM final) was added to a pre-warmed (10 min, 37° C.) reaction mixture (60 μL final) containing Na-benzoyl arginine ethyl ester (BAEE, 10 mM), CaCl₂ (10 mM), TRIS-HCl (100 mM, pH 7.4), NaCl (50 mM) and DTT (2 mM). This mixture was incubated at 37° C. for 0, 30, 50 and 90 min after which the reaction was stopped by flash freezing with liquid nitrogen. The production of citrulline at various time points was quantitated by the COLDER assay. (Kearney, et al. Biochemistry 44, 10570-10582 (2005); Knipp, et al. Anal Biochem 286, 257-264 (2000).) The time-dependent production of citrulline by PAD4 was fit to the equation for a straight line using Graphpad Prism. All the reactions were performed at least in duplicate.

### Michaelis-Menten Kinetics of Wild-Type and Mutant PAD4

PAD4 (50 nM final for wild-type, 75 nM final for R372Cit, and 100 nM final for R374Cit) was added to a pre-warmed (10 min, 37° C.) reaction mixture (60 μL final) containing various concentrations of BAEE (0, 0.5, 1, 2.5, 5 and 10 mM), CaCl₂ (10 mM), TRIS-HCl (100 mM, pH 7.4), NaCl (50 mM) and DTT (2 mM). This mixture was incubated at 37° C. for 45 min (for wild-type), 90 min (for R374Cit) and 120 min (for R372Cit) followed by quenching with liquid nitrogen. The rate of citrulline formation at various BAEE concentrations was quantified with the COLDER assay. The rates were plotted against the BAEE concentration and were fit to the Michaelis-Menten equation using Graphpad Prism. All the reactions were performed at least in duplicate.

### Calcium-Dependence of Wild-Type PAD4

PAD4_{Bac} (purified from bacterial expression system) and PAD4_{Mam} (purified from mammalian expression system) (6 μL of 0.5 μM stock, 50 nM final) were added to separate pre-warmed (10 min, 37° C.) reaction mixtures (60 μL final) containing BAEE (10 mM), various concentrations of CaCl₂ (0, 0.25, 0.5, 1, 2.5, 5 and 10 mM), TRIS-HCl (100 mM, pH 7.4), NaCl (50 mM) and DTT (2 mM). This mixture was incubated at 37° C. for 45 min followed by flash freezing with liquid nitrogen. The production of citrulline at various concentration of CaCl₂ by PAD4 was quantified with the COLDER assay. (Tanikawa, et al. 2012 Nat. Commun. 3, 676; Zhang, et al. 2012 Proc. Natl. Acad. Sci. USA 109, 13331-13336.)

The calcium-dependence of citrulline production by PAD4 was fit to the equation 2,

v=Vₘₐₓ*[Ca²⁺]^{h}/(K_{0.5}^{h}+[Ca²⁺]^{h}) (2)

using Graphpad Prism, where v is the velocity of the reaction, Vₘₐₓ is the maximum velocity of the reaction, [Ca²⁺] is the concentration of calcium, h is the hill slope and K_{0.5} is the calcium concentration that gives half-maximal velocity. All the reactions were performed at least in duplicate.

### Effect of Autocitrullination of Wild-Type PAD4 on the Enzymatic Activity

PAD4_{Bac} (0.5 μM final) was added to a pre-warmed (10 min, 37° C.) solution containing CaCl₂ (0 or 10 mM), TRIS-HCl (100 mM, pH 7.4), NaCl (500 mM), DTT (2 mM). At various time points (0, 5, 15, 30, 60 and 90 min), 6 μL of this reaction mixture was removed and was added to a pre-warmed (10 min, 37° C.) reaction mixture (10 mM BAEE, 10 mM CaCl₂, 100 mM TRIS, pH 7.4, 500 mM NaCl, 2 mM DTT, with a final volume of 60 μL). After 45 min, the reaction mixture was flash frozen with liquid nitrogen. The production of citrulline at various time points was quantified with the COLDER assay. The loss in activity of PAD4 over time was fit into single exponential decay using Graphpad Prism. All the reactions were performed at least in duplicate.

### Rhodamine-PG Labeling of Autocitrullinated PAD4

Rhodamine-PG labelling of autocitrullinated PAD4 was performed as reported earlier with minor modifications. (Bicker, et al. 2012 J. Am. Chem. Soc. 134, 17015-17018.) PAD4 (0.5 μM final) was added to a pre-warmed (10 min, 37° C.) reaction mixture containing CaCl₂ (0 or 10 mM), TRIS-HCl (100 mM, pH 7.4), NaCl (500 mM), DTT (2 mM). At various time points (0, 5, 15, 30, 60 and 90 min), the reaction was stopped by flash freezing in liquid nitrogen. The samples were thawed, and trichloroacetic acid (20% final) and rhodamine-PG (80 μM final) were sequentially added to it. The reaction mixture was incubated at 37° C. for 1 h. Then the reaction was quenched with citrulline (100 mM final) dissolved in 50 mM TRIS-HCl (pH 7.4) and the mixture was further incubated at 37° C. for 30 min. The samples were placed at −20° C. for 30 min and the precipitated proteins were collected by centrifugation (15000 rpm for 30 min) at 4° C. The protein pellet was washed with cold acetone and dried. Then the pellet was dissolved in 40 μL buffer containing 100 mM arginine, 20 mM TRIS-HCl (pH 7.4), 1% SDS and 10 μL 5×SDS loading dye. The proteins were separated by SDS-PAGE and the fluorescently labelled bands were visualized by scanning the gel in a typhoon scanner (excitation and emission maxima ˜546 and 579 nm, respectively). The fluorescent intensities of protein bands were quantified using ImageJ software and were normalized against the coomassie intensities that indicate the amount of protein present in each lane. All the reactions were performed at least in duplicate.

### RFA Labelling of Wild-Type and R374Cit PAD4

RFA labelling of PAD4 was carried out by following a protocol similar to that established for PAD1 and PAD2. (Mondal, et al. Angew. Chem. Int. Ed. 58, 12476-12480 (2019); Mondal, et al. ACS Chem. Biol. 13, 1057-1065 (2018).) Briefly, PAD4 (100 nM final) was added to pre-warmed (10 min, 37° C.) reaction mixture (100 mM TRIS pH 7.4, 500 mM NaCl, 0 or 10 mM CaCl2, and 2 mM DTT in a final volume of 30 μL) containing RFA (200 nM final). After incubating at 37° C. for 2 h, the reaction mixture was quenched with 5×SDS-PAGE loading dye and boiled at 95° C. for 10 min. The proteins were separated by SDS-PAGE using a 4-20% gradient gel and fluorescently labelled proteins were visualized by scanning the gel in a typhoon scanner (excitation and emission maxima ˜546 and 579 nm, respectively). The fluorescent intensities of protein bands were quantified using ImageJ software. All the reactions were performed at least in duplicate.

### RFA Labelling of Wild-Type and R374Cit PAD4 in EXPI293F Lysate

RFA (10 μM) was added to a pre-warmed (10 min, 37° C.) reaction mixture (2 mg/mL EXPI293F lysate containing wild-type or R374Cit or R372Cit PAD4 in 1×PBS, 2 mM CaCl2, and 2 mM DTT in a final volume of 50 μL) and the mixture was incubated at 37° C. for 2 h. The reaction was quenched with 5×SDS-PAGE loading dye and was boiled at 95° C. for 10 min. The proteins were separated on a 4-20% SDS-PAGE gel and the fluorescently labelled proteins were visualized by scanning the gel in a typhoon scanner (excitation and emission maxima ˜546 and 579 nm, respectively). The fluorescent intensities of protein bands were quantified using ImageJ software. All the reactions were performed at least in duplicate.

### Histone H3 Citrullination in Live HEK293T Cells

HEK293T cells were seeded (4×10⁵ cells/well) on 6-well plates and were allowed to grow in DMEM (supplemented with 10% heat-inactivated fetal bovine serum, 100 units/mL penicillin, 100 μg/mL streptomycin) for 48 h. Cells were then transfected with appropriate plasmids (3 μg PAD4 plasmid and 2 μg pIDTSMART eRF1 E55D for each well) using Lipofectamine 2000 (12.5 μg for each well) in Opti-MEM medium (Gibco). After 5 h, the medium was changed to DMEM and DMSO (for WT PAD4) or SM60 (1 mM for R374Cit PAD4) was added. Cells were then allowed to grow at 37° C. for 48 h. Cells were washed with serum-free DMEM (supplemented with 100 units/mL penicillin, 100 mg/mL streptomycin), resuspended in the same medium and were irradiated with 365 nm UV for 3 min. Then the cells were treated with CaCl₂ (1 mM) and a combination of CaCl₂ (1 mM) and ionomycin (5 μM) for 3 h at 37° C. After this, cells were lysed and the lysates were analyzed by Western blotting using anti-histone H3 citrulline (R2,8,17) and anti-histone H3 primary antibodies. PAD4 expression was quantified by using an anti-PAD4 primary antibody. Band intensities for citrullinated histone H3 were normalized against those for histone H3 and PAD4 using the following algorithm,

Normalized histone H3Cit=[H3Cit/(H3_33 PAD4)]×10⁶

All the experiments were performed in triplicate.

### Thermal Shift Assay

A 50 μL reaction mixture (EXPI293F lysate containing overexpressed wild-type PAD4 (1.5 mg/mL) or R374Cit mutant (2 mg/mL), 1×PBS and DMSO (1% final)) was heated at various temperatures (25, 40, 50, 60, 65, 70, 75, 85 and 90° C.) for 5 min followed by flash freezing with liquid nitrogen. The samples were thawed and the precipitated proteins were separated by centrifugation (15000 rpm, 30 min) at 4° C. 40 μL of the supernatant was mixed with 10 μL of 5×SDS-PAGE loading dye and the mixture was boiled at 95° C. for 10 min. The proteins were separated by SDS-PAGE using a 4-20% gradient gel and the soluble fractions of PAD4 were quantitated by western blot analysis using a rabbit polyclonal anti-PAD4 antibody. This assay was also performed separately in the presence of CaCl₂ (1 mM final) and a PAD4-selective ligand, GSK199 (10 μM final). For these assays, the reaction mixture was incubated with CaCl₂ or GSK199 at room temperature for 5 min before heating up at different temperatures. All the reactions were performed at least in duplicate.

### Proteomics Study on Autocitrullinated PAD4

50 μg PAD4 was incubated at 37° C. for various times (0, 5, 15, 30 and 90 min) in the absence and presence (10 mM) of CaCl₂ followed by flash freezing with liquid nitrogen. The samples were thawed and trichloroacetic acid (20% final) was added. Then the samples were placed at −20° C. for 30 min and the precipitated proteins were collected by centrifugation (15000 rpm, 30 min) at 4° C., washed with cold acetone, and dried. The protein pellet was resuspended in 6 M urea (100 μL) in PBS and TCEP (2 mM final) was added to it. The solution was incubated at 37° C. for 1 h. Then iodoacetamide (4 mM final) was added and the solution was incubated at 37° C. for 30 min in dark. Then 200 μL of PBS was added to the solution to achieve a final concentration of urea of 2 M. Lys-C (1 μL of a 1 μg/μL solution, reconstituted in water) and Glu-C (2 μL of a 0.5 μg/μL solution, reconstituted in water) were then added to the samples and then incubated at 37° C. for 16 h. The proteolysis reaction was terminated by adding 15 μL of formic acid. The peptide mixture was then desalted with Pierce™ desalting C18 spin columns, resuspended in 120 μL of HEPES buffer (100 mM, pH 8.5) and the total peptide content in each sample was quantified and normalized using the Pierce™ Quantitative Fluorometric Peptide Assay kit, according to the manufacturer's protocol. 8 μL of a 19.5 μg/μL acetonitrile stock of TMT10plex™ isobaric labelling reagents (TMT10-126, TMT10-127N, TMT10-127C, TMT10-128N, TMT10-128C, TMT10-129N, TMT10-129C, TMT10-130N, TMT10-130C and TMT10-131) were added to 100 μL samples treated in the absence and presence of calcium for 5 different time points (0, 5, 15, 30 and 90 min). The reaction mixtures were incubated at room temperature for 1 h. To quench the reaction, 8 μL of 5% hydroxylamine was added to each sample and the mixture was incubated at room temperature for 15 min. Then the 10 samples (0, 5, 15, 30 and 90 min samples in the absence and presence of calcium) were combined together in a new microcentrifuge tube and desalted using C18 spin columns. This experiment was performed in triplicate.

### LC-MS/MS Analysis

Peptides were lyophilized, resuspended in 5% acetonitrile, 0.1% (v/v) formic acid in water, and loaded at 4.0 μL/min by a NanoAcquity UPLC (Waters Corporation, Milford, MA) onto a 100 μm I.D. fused-silica pre-column packed with 2 cm of 5 μm (200 Å) Magic C18AQ (Bruker-Michrom), equilibrated with 5% acetonitrile, 0.1% (v/v) formic acid in water. After trapping for 4.0 minutes on the pre-column, peptides were eluted at 300 nL/min from a 75 μm I.D. gravity-pulled analytical column packed with 25 cm of 3 μm (100 Å) Magic C18AQ particles using a gradient of mobile phase A, 0.1% (v/v) formic acid in water and mobile phase B, 0.1% (v/v) formic acid in acetonitrile as follows; 0-100 min (5-35% B), 100-120 min (35-65% B), 120-121 min (65-95% B), and 121-126 min (95% B). Ions were introduced by positive electrospray ionization via liquid junction at 1.4 kV into a Q Exactive hybrid quadrapole orbitrap mass spectrometer (Thermo Scientific, Waltham, MA). Mass spectra were acquired over m/z 300-1750 at 70,000 resolution (m/z 200) with an AGC target of 1e6, and data-dependent acquisition selected the top 10 most abundant precursor ions for tandem mass spectrometry by HCD fragmentation using an isolation width of 1.6 Da, max fill time of 100 ms, and AGC target of 1e5. Peptides were fragmented by a normalized collisional energy (NCE) of 27 and product ion spectra acquired at a resolution of 17500 (m/z 200). For TMT-labeled samples, NCE was set to 32 and product ion spectra were acquired at a resolution of 35000 (m/z 200).

### LC-MS/MS Data Analysis

Raw data files were peak processed with Proteome Discoverer (version 2.1, ThermoScientific, Waltham, MA) followed by identification using Mascot Server (version 2.5, Matrix Science) against the Swissprot human or E. coli (TMT-labeled samples) FASTA file. Proteolytic enzyme was set to LysC and GluC with two missed cleavages. Variable modifications of N-terminal acetylation, oxidized methionine, pyroglutamic acid for glutamine, deamidation of asparagine, and citrullination of arginine were implemented. Carbamidomethylation of cysteines and TMT6-plex modification at lysine and peptide N-terminus were set as fixed modifications. Assignments were made using a 10 ppm mass tolerance for the precursor and 0.05 Da mass tolerance for the fragments. All non-filtered search results were processed by Scaffold (version 4.10.0, Proteome Software, Inc.) utilizing the Trans-Proteomic Pipeline (Institute for Systems Biology) with threshold values set at 90% for peptides (1% false-discovery rate) and 99% for proteins (2 peptides minimum, 6% false-discovery rate). TMT product ion ratios were calculated using the Scaffold Q+S analysis software.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound having the structural formula (I): 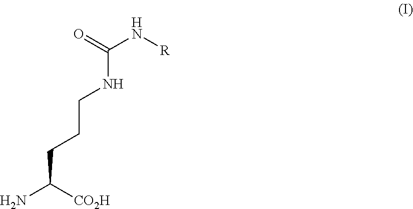
wherein R is a photoreleasable group.

2. The compound of claim 1, wherein R is: 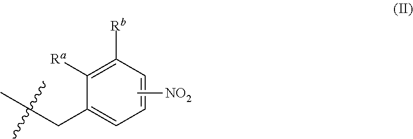
wherein each of R^{a} and R^{b} is selected from H or OR^{c}, wherein R^{c} is a C₁-C₆ alkyl group.

3. The compound of claim 2, wherein R is:

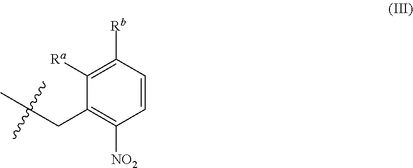

4. The compound of claim 1, wherein R is:

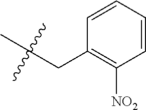 [SMILES: *Cc1ccccc1[N+](=O)[O-]]

5. The compound of claim 1, wherein R is:

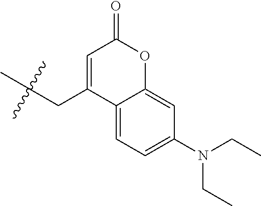 [SMILES: *Cc1cc(=O)oc2cc(N(CC)CC)ccc12]

6. The compound of claim 1, wherein R is:

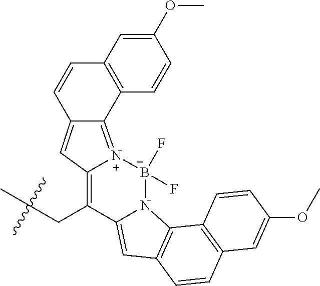 [SMILES: *CC1=C2C=c3ccc4cc(OC)ccc4c3=[N+]2[B-](F)(F)n2c1cc1ccc3cc(OC)ccc3c12]

7. A peptide comprising the compound of claim 1.

8. A method for treating a disease comprising administering to a patient in need thereof the peptide of claim 7.

9. A pharmaceutical composition comprising a peptide of claim 7 and a pharmaceutically acceptable excipient, carrier or diluent.

10. A method for treating a disease comprising administering to a patient in need thereof the pharmaceutical composition of claim 9.

11. A method for site-specific citrullination in a synthetic peptide, comprising:
incorporating an unnatural citrulline analog of claim 1 at one or more positions in the synthetic peptide where citrullination is desired; and
photochemically converting the unnatural citrulline analog to citrulline.

12. The method of claim 11, wherein incorporating an unnatural citrulline analog is achieved by using an E. coli-derived engineered leucyl-tRNA synthetase (EcLeuRS)/tRNA_{CUA}^{EcLeu} pair.

13. The method of claim 12, wherein photochemical conversion of the unnatural citrulline analog to citrulline comprises directing a UV irradiation at the synthetic peptide.

14. The method of claim 13, wherein the UV-Visible irradiation comprises wavelengths in the range of about 365 nm to about 700 nm.

15. The method of claim 14, wherein the UV irradiation comprises the wavelength of 365 nm.

16. The method of claim 11, wherein the photochemical conversion results in greater than 95% conversion of the unnatural citrulline analog to citrulline.
